(19) United States
(12) Patent Application Publication
(10) Pub. No.: US 2018/0141922 A1
(43) Pub. Date: May 24, 2018

(54) BENZOXAZINE DERIVATIVES AND USE IN HAIR DYEING

(71) Applicant: L'OREAL, Paris (FR)

(72) Inventor: Aziz FADLI, Chelles (FR)

(21) Appl. No.: 15/502,590

(22) PCT Filed: Aug. 6, 2015
(86) PCT No.: PCT/EP2015/068191

§ 371 Date: Feb. 8, 2017

(30) Foreign Application Priority Data
Aug. 8, 2014 (FR) 1457694

(51) Int. Cl. C07D 265/36; C07D 401/06; C07D 413/06; A61K 8/49; A61Q 5/10

(52) U.S. Cl. CPC C07D 265/36; C07D 401/06; A61Q 5/10; A61K 8/49; C07D 413/06

(57) ABSTRACT

The invention relates to the use of a compound of formula (I) below, addition salts thereof, optical isomers, geometrical isomers and tautomers thereof and/or solvates thereof: Formula (I) in which: • R represents a hydrogen atom or a C1-C4 alkyl or C1-C4 hydroxyalkyl radical, preferably a hydrogen atom, • R₂, R₃, R₄ and R₅ independently represent: —a hydrogen atom, —a linear C1-C4 or branched C3-C4 alkyl radical, optionally substituted with a hydroxyl radical, • R₁ represents a linear C2-C10 or branched C3-C10 alkyl radical, optionally interrupted with one or more heteroatoms chosen from O or S or with one or more groups —NR′, said alkyl radical ending with a group —NX₁X₂ or —OX₃ or a radical chosen from: Formula (II) —X₁ and X₂ independently denote ◯ a hydrogen atom ◯ a linear C1-C6 alkyl radical or a branched C3-C6 alkyl radical ◯ a linear C1-C6 hydroxyalkyl radical or a branched C3-C6 hydroxyalkyl radical, X₁ and X₂ may form, with the nitrogen atom that bears them, a saturated or unsaturated 5- to 8-membered heterocycle, in which one of the ring members may be a heteroatom chosen from O, S and N; said heterocycle possibly being substituted with one or more linear or branched C₁-C₄ alkyl, C₁-C₄ hydroxyalkyl or dimethylamino radicals, —X₃ denotes a hydrogen atom or a linear C1-C6 or branched C3-C6 alkyl radical, —R′ denotes a hydrogen atom or a linear C1-C4 or branched C3-C4 alkyl radical, for dyeing keratin fibres, especially human keratin fibres such as the hair.

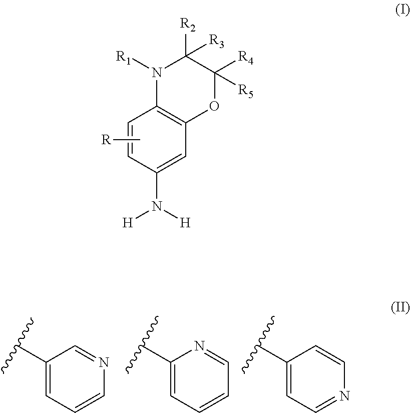

The invention relates to the use, for the dyeing of keratin fibres, of benzoxazine-based compounds, and also to a dyeing process using these compounds.

It is known practice to dye keratin fibres and in particular human hair with dye compositions containing oxidation dye precursors, which are generally known as oxidation bases, such as ortho- or para-phenylenediamines, ortho- or para-aminophenols and heterocyclic compounds. These oxidation bases are colourless or weakly coloured compounds, which, when combined with oxidizing products, may give rise to coloured compounds via a process of oxidative condensation.

It is also known that the shades obtained with these oxidation bases may be varied by combining them with couplers or colour modifiers, the latter being chosen especially from aromatic meta-diamines, meta-aminophenols, meta-diphenols and certain heterocyclic compounds such as indole compounds.

The variety of molecules used as oxidation bases and couplers allows a wide range of colours to be obtained.

The “permanent” colouring obtained by means of these couplers and oxidation dyes must moreover satisfy a certain number of requirements. Thus, it should have no toxicological drawbacks, it should allow shades to be obtained in the desired intensity, and it should show good resistance to external agents such as light, bad weather, washing, permanent waving treatments, perspiration and rubbing.

The dyes should also allow grey hair to be covered and, finally, they should be as unselective as possible, i.e. they should produce the smallest possible differences in colour along the same keratin fibre, which in general is differently sensitized (i.e. damaged) between its end and its root.

The aim of the present invention is to obtain hair dyeing compounds that can afford improved dyeing properties in terms of intensity or chromaticity and/or selectivity and/or resistance to external agents.

Surprisingly and advantageously, the Applicant has just discovered a new family of oxidation dye precursors consisting of benzoxazine derivatives. These compounds result in a wide range of colours in oxidation dyeing. They especially make it possible to broaden the colour range, and to obtain powerful, chromatic and sparingly selective colourings in varied shades, which show good resistance to the various external attacking factors to which the hair may be subjected (shampoo, light, sweat or permanent reshaping).

One subject of the invention is thus the use of a compound of formula (I) below, addition salts thereof, optical isomers, geometrical isomers and tautomers thereof and/or solvates thereof:

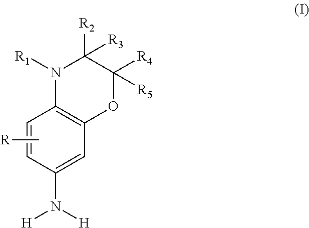

with:
R represents a hydrogen atom or a C1-C4 alkyl or C1-C4 hydroxyalkyl radical; preferably, R represents a hydrogen atom,
R₂, R₃, R₄ and R₅ independently represent:
a hydrogen atom,
a linear C1-C4 or branched C3-C4 alkyl radical, optionally substituted with a hydroxyl radical,
R₁ represents a linear C2-C10 or branched C3-C10 alkyl radical, optionally interrupted with one or more non-adjacent heteroatoms chosen from O or S or with one or more non-adjacent groups —NR′, said alkyl radical ending with a group —NX₁X₂ or —OX₃ or a radical chosen from:

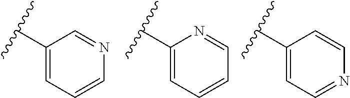

X₁ and X₂ independently denote
   a hydrogen atom,
   a linear C1-C6 alkyl radical or a branched C3-C6 alkyl radical,
   a linear C1-C6 hydroxyalkyl radical or a branched C3-C6 hydroxyalkyl radical,

X₁ and X₂ may form, with the nitrogen atom that bears them, a saturated or unsaturated 5- to 8-membered heterocycle, in which one of the ring members may be a heteroatom chosen from O, S and N; said heterocycle possibly being substituted with one or more linear or branched C₁-C₄ alkyl, C₁-C₄ hydroxyalkyl or dimethylamino radicals,
X₃ denotes a hydrogen atom or a linear C1-C6 or branched C3-C6 alkyl radical,
R′ denotes a hydrogen atom or a linear C1-C4 or branched C3-C4 alkyl radical,

with the exception of the following compounds:

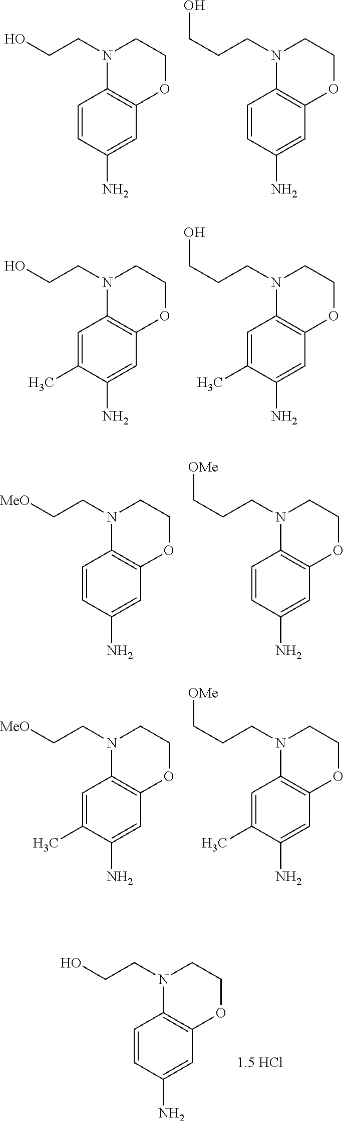

for dyeing keratin fibres, especially human keratin fibres such as the hair.

A subject of the invention is also the use of a compound of formula (I′) below, addition salts thereof, optical isomers, geometrical isomers and tautomers thereof and/or solvates thereof:

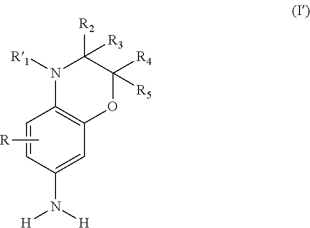

with:
R represents a hydrogen atom or a C1-C4 alkyl or C1-C4 hydroxyalkyl radical. Preferably, R represents a hydrogen atom,
R_{2,} R₃, R₄ and R₅ independently represent:
   a hydrogen atom,
   a linear C1-C4 or branched C3-C4 alkyl radical, optionally substituted with a hydroxyl radical,
R′₁ represents a radical X₄X₅N-Alk-,
Alk represents a linear or branched divalent C2-C10 alkyl radical, optionally interrupted with one or more non-adjacent heteroatoms chosen from O and S or with one or more non-adjacent groups —NR′ in which R′ denotes a hydrogen atom or a C1-C4 alkyl radical; preferably, Alk denotes a divalent C2-C5 alkyl radical optionally interrupted with an oxygen atom or with an —NH group,
X₄ and X₅ independently denote:
   a hydrogen atom,
   a linear C1-C6 alkyl radical or a branched C3-C6 alkyl radical, optionally ending with a radical —OX₆, X₆ denoting a hydrogen atom or a linear C1-C6 or branched C3-C6 alkyl radical,
X₄ and X₅ may form, with the nitrogen atom that bears them, a saturated or unsaturated 5- to 8-membered heterocycle, in which one of the ring members may be a heteroatom chosen from O, S and N; said heterocycle possibly being substituted with one or more linear or branched C₁-C₄ alkyl, C₁-C₄ hydroxyalkyl or dimethylamino radicals,

for dyeing keratin fibres, especially human keratin fibres such as the hair.

According to a third subject, the invention relates to a benzoxazine-based compound chosen from:
i) compounds of formula (II) below, addition salts thereof, optical isomers, geometrical isomers and tautomers thereof and/or solvates thereof:

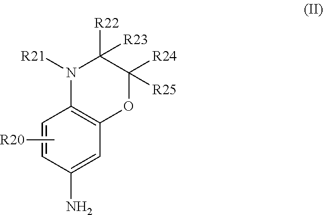

with:
R₂₁ represents a radical chosen from:
   a linear C4-C10 alkyl radical ending with a group —NX₂₁X₂₂ or —OX₂₃,
   a branched C3-C10 alkyl radical ending with a group —NX₂₁X₂₂ or —OX₂₃,
   a linear C2-C10 or branched C3-C10 alkyl radical, interrupted with one or more non-adjacent heteroatoms chosen from O and S or with one or more non-adjacent groups —NR′₂₀, said alkyl radical ending with a group —NX₂₁X₂₂ or —OX₂₃ or
   a linear C2-C3 alkyl radical ending with a group —NX₃₁X₃₂ or —OX₃₃,
   a linear C2-C10 or branched C3-C10 alkyl radical, optionally interrupted with one or more non-adjacent heteroatoms chosen from O and S or with one or more non-adjacent groups —NR′₂₀, said alkyl radical ending with a radical chosen from

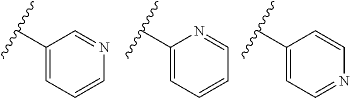

X₂₁ and X₂₂ independently denote:
   a hydrogen atom
   a linear C1-C6 alkyl radical or a branched C3-C6 alkyl radical
   a linear C1-C6 hydroxyalkyl radical or a branched C3-C6 hydroxyalkyl radical,
X₂₁ and X₂₂ may form, with the nitrogen atom that bears them, a saturated or unsaturated 5- to 8-membered heterocycle, in which one of the ring members may be a heteroatom chosen from O, S and N; said heterocycle possibly being substituted with one or more linear or branched C₁-C₄ alkyl, C₁-C₄ hydroxyalkyl or dimethylamino radicals,
X₂₃ denotes a hydrogen atom or a linear C1-C6 or branched C3-C6 alkyl radical, preferably a hydrogen atom
R′₂₀ denotes a hydrogen atom or a linear C1-C4 or branched C3-C4 alkyl radical,
X₃₁ and X₃₂ independently denote:
   a linear C2-C6 alkyl radical or a branched C3-C6 alkyl radical
   a linear C1-C6 hydroxyalkyl radical or a branched C3-C6 hydroxyalkyl radical
X₃₃ denotes a linear C2-C6 or branched C3-C6 alkyl radical
R₂₀ represents a hydrogen atom or a C1-C4 alkyl or C1-C4 hydroxyalkyl radical. Preferably, R₂₀ represents a hydrogen atom,
R₂₂, R₂₃, R₂₄ and R₂₅ independently represent a hydrogen atom or a linear C1-C4 or branched C3-C4 alkyl radical optionally substituted with a hydroxyl radical,
ii) the following compounds

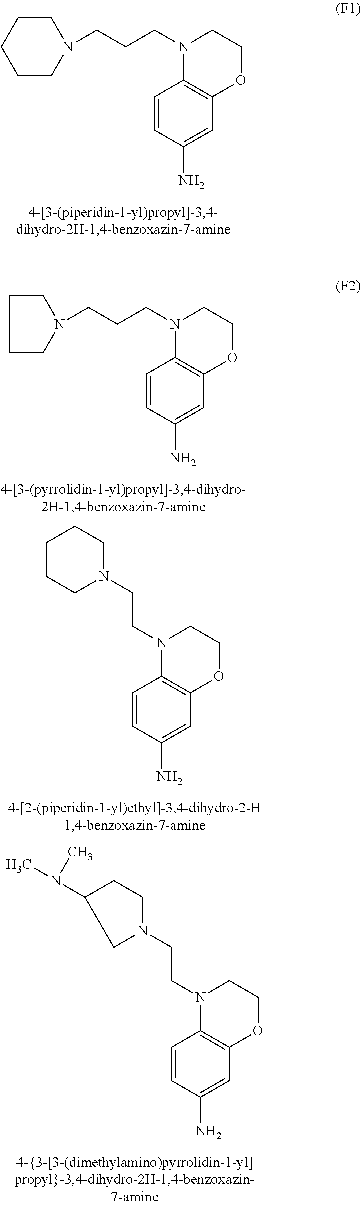 [SMILES: Nc1ccc2c(c1)OCCN2CCCN1CCCCC1 ; Nc1ccc2c(c1)OCCN2CCCN1CCCC1 ; Nc1ccc2c(c1)OCCN2CCN1CCCCC1 ; CN(C)C1CCN(CCN2CCOc3cc(N)ccc32)C1]

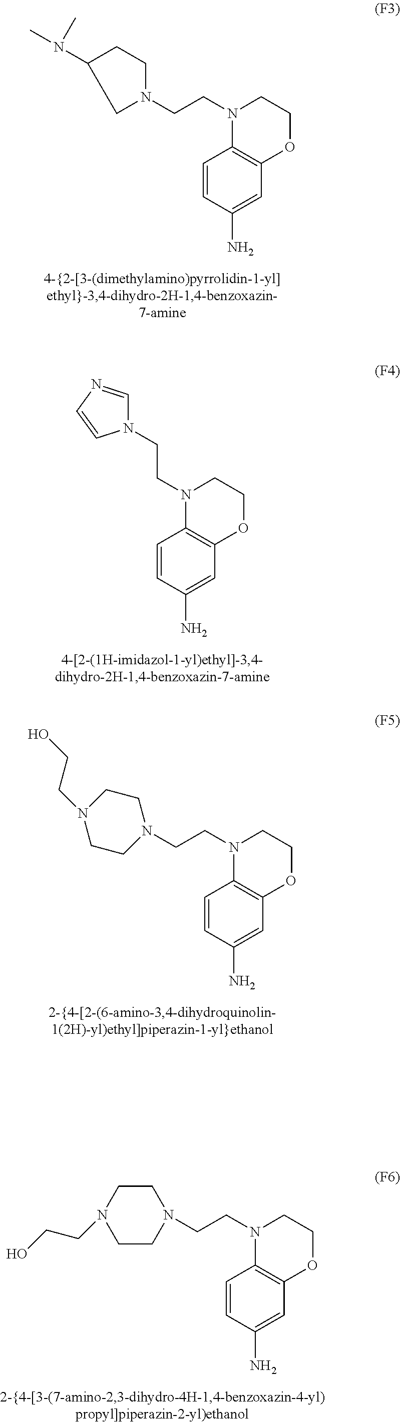 [SMILES: CN(C)C1CCN(CCN2CCOc3cc(N)ccc32)C1 ; Nc1ccc2c(c1)OCCN2CCn1ccnc1 ; Nc1ccc2c(c1)OCCN2CCN1CCN(CCO)CC1 ; Nc1ccc2c(c1)OCCN2CCN1CCN(CCO)CC1]

and also the addition salts thereof, optical isomers, geometrical isomers and tautomers thereof and/or solvates thereof.

A subject of the invention is also a composition, especially a cosmetic composition and in particular a composition for treating keratin fibres such as human keratin fibres and in particular the hair, comprising at least one benzoxazine-based compound chosen from the compounds of formula (II) and/or compounds F1 to F6 described above.

A subject of the invention is also the use of a benzoxazine-based compound chosen from the compounds of formula (II) and/or compounds F1 to F6 as defined above, for dyeing keratin fibres, especially human keratin fibres such as the hair.

A subject of the invention is also a process for dyeing keratin fibres, which consists in applying to said fibres a composition comprising a compound of formula (I), (I′), (II) and/or F1 to F6 described previously.

The compounds of the present invention make it possible in particular to obtain compositions for dyeing keratin fibres that are suitable for use in oxidation dyeing and that make it possible to obtain a hair colouring that has improved dyeing properties in terms of intensity or chromaticity and/or selectivity and/or resistance to external agents such as shampoo, sweat, permanent reshaping and light.

The compounds of general formulae (I), (I′), (II) and F1 to F6 may be in free form or in the form of salts, such as addition salts with a mineral acid preferably chosen from hydrochloric acid, hydrobromic acid, sulfuric acid and phosphoric acid or with an organic acid such as, for example, citric acid, succinic acid, tartaric acid, lactic acid, 4-tolylsulfonic acid, benzenesulfonic acid, acetic acid, para-toluenesulfonic acid, formic acid and methanesulfonic acid.

These compounds may also be in the form of solvates, for example a hydrate or a solvate of a linear or branched alcohol such as ethanol or isopropanol.

For the purposes of the present invention, and unless otherwise indicated:
an “alkyl radical” is a linear or branched C₁-C₂₀ and preferably C₁-C₈ hydrocarbon-based radical;
an “alkoxy radical” is an alkyl-oxy radical for which the alkyl radical is a linear or branched C₁-C₁₆ and preferentially C₁-C₈ hydrocarbon-based radical;
when the alkoxy group is optionally substituted, this implies that the alkyl group is optionally substituted as defined hereinabove;
the term “at least one” is equivalent to the term “one or more”; and
the term “inclusively” for a range of concentrations means that the limits of that range are included in the defined range.

It should be noted that, in the text hereinbelow, unless otherwise indicated, the limits of a range of values are included in that range.

According to a first subject, the invention thus relates to the use of a compound of formula (I) below, addition salts thereof, optical isomers, geometrical isomers and tautomers thereof and/or solvates thereof:

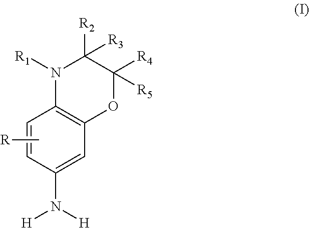

with:
R represents a hydrogen atom or a C1-C4 alkyl or C1-C4 hydroxyalkyl radical. Preferably, R represents a hydrogen atom,
R_{2,} R₃, R₄ and R₅ independently represent:
   a hydrogen atom,
   a linear C1-C4 or branched C3-C4 alkyl radical, optionally substituted with a hydroxyl radical,
R₁ represents a linear C2-C10 or branched C3-C10 alkyl radical, optionally interrupted with one or more non-adjacent heteroatoms chosen from O or S or with one or more non-adjacent groups —NR′, said alkyl radical ending with a group —NX₁X₂ or —OX₃ or a radical chosen from:

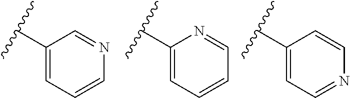

X₁ and X₂ independently denote
   a hydrogen atom
   a linear C1-C6 alkyl radical or a branched C3-C6 alkyl radical o a linear C1-C6 hydroxyalkyl radical or a branched C3-C6 hydroxyalkyl radical,

X₁ and X₂ may form, with the nitrogen atom that bears them, a saturated or unsaturated 5- to 8-membered heterocycle, in which one of the ring members may be a heteroatom chosen from O, S and N; said heterocycle possibly being substituted with one or more linear or branched C₁-C₄ alkyl, C₁-C₄ hydroxyalkyl or dimethylamino radicals
X₃ denotes a hydrogen atom or a linear C1-C6 or branched C3-C6 alkyl radical, preferably a hydrogen atom
R′ denotes a hydrogen atom or a linear C1-C4 or branched C3-C4 alkyl radical,

with the exception of the following compounds:

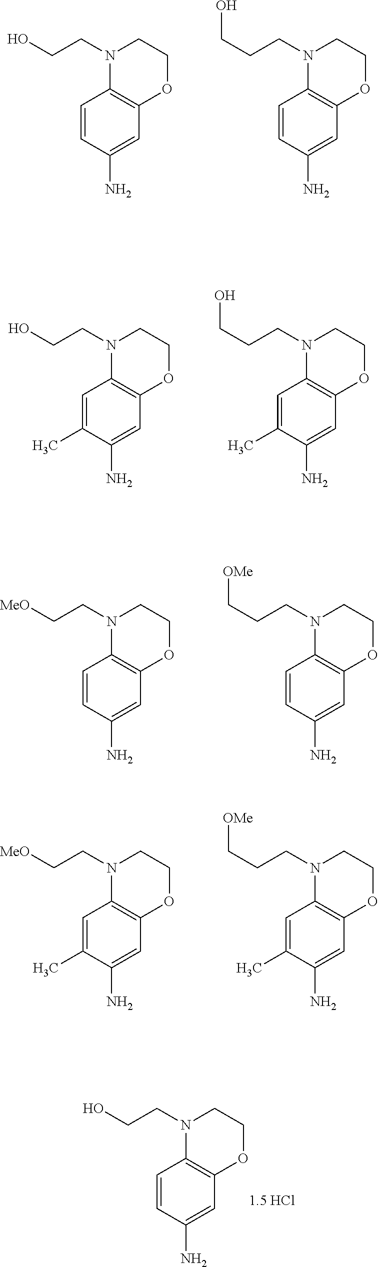

for dyeing keratin fibres, especially human keratin fibres such as the hair.

As examples of saturated or unsaturated 5- to 8-membered heterocycles in which one of the ring members may be a heteroatom chosen from O, S and N, mention may be made of imidazole, pyridine, piperazine, pyrrolidine, morpholine, pyrimidine, thiazole, benzimidazole, benzothiazole, oxazole, benzotriazole, triazole, pyrazole, benzoxazole and piperidine rings.

Preferably, said saturated or unsaturated 5- to 8-membered heterocycle in which one of the ring members may be a heteroatom chosen from O, S and N is chosen from imidazole, piperazine, pyrrolidine, morpholine and piperidine rings.

Preferably, R, R₂, R₃, R₄ and R₅ represent a hydrogen atom.

Preferably, R₁ represents a linear C2-C10 or branched C3-C10 alkyl radical, optionally interrupted with a heteroatom chosen from O and S and/or with a group —NH, said alkyl radical ending with a group —NX₁X₂ or —OX₃.

Preferably also, R1 represents a linear C2-C6 or branched C3-C6 alkyl radical, optionally interrupted with an oxygen atom and/or with a group —NH, said alkyl radical ending with a group —NX₁X₂ or —OX₃, X₁, X₂ and X₃ being as defined above.

Preferably, X₃ denotes a hydrogen atom or a linear C1-C4 or branched C3-C4 alkyl radical. More preferably, X3 denotes a hydrogen atom.

### I/ According to a First Embodiment of the Invention, the Compounds of Formula (I) are such That

R, R₂, R₃, R₄ and R₅ represent a hydrogen atom,

R₁ represents a linear C2-C10 or branched C3-C10 alkyl radical, ending with a group —NX₁X₂

X₁ and X₂ independently denote:
a hydrogen atom
a linear C1-C6 alkyl radical or a branched C3-C6 alkyl radical
a linear C1-C6 hydroxyalkyl radical or a branched C3-C6 hydroxyalkyl radical,

X₁ and X₂ may form, with the nitrogen atom that bears them, a saturated or unsaturated 5- to 8-membered heterocycle, in which one of the ring members may be a heteroatom chosen from O, S and N; said heterocycle possibly being substituted with one or more linear or branched C₁-C₄ alkyl, C₁-C₄ hydroxyalkyl or dimethylamino radicals.

As examples of saturated or unsaturated 5- to 8-membered heterocycles in which one of the ring members may be a heteroatom chosen from O, S and N, mention may be made of imidazole, pyridine, piperazine, pyrrolidine, morpholine, pyrimidine, thiazole, benzimidazole, benzothiazole, oxazole, benzotriazole, triazole, benzoxazole and piperidine rings.

Preferably, said saturated or unsaturated 5- to 8-membered heterocycle in which one of the ring members may be a heteroatom chosen from O, S and N, is chosen from imidazole, piperazine, pyrrolidine, morpholine and piperidine rings, which may be optionally substituted with one or more linear or branched C₁-C₄ alkyl, C₁-C₄ hydroxyalkyl or dimethylamino radicals.

According to this first variant, the compounds that are particularly preferred are the following:

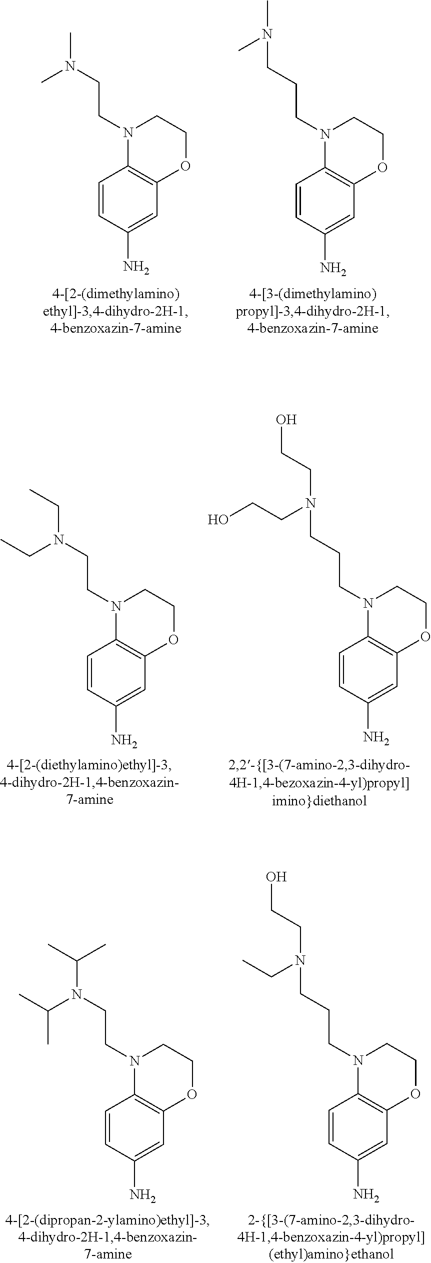 [SMILES: CN(C)CCN1CCOc2cc(N)ccc21 ; CN(C)CCCN1CCOc2cc(N)ccc21 ; CCN(CC)CCN1CCOc2cc(N)ccc21 ; Nc1ccc2c(c1)OCCN2CCCN(CCO)CCO ; CC(C)N(CCN1CCOc2cc(N)ccc21)C(C)C ; CCN(CCO)CCCN1CCOc2cc(N)ccc21]

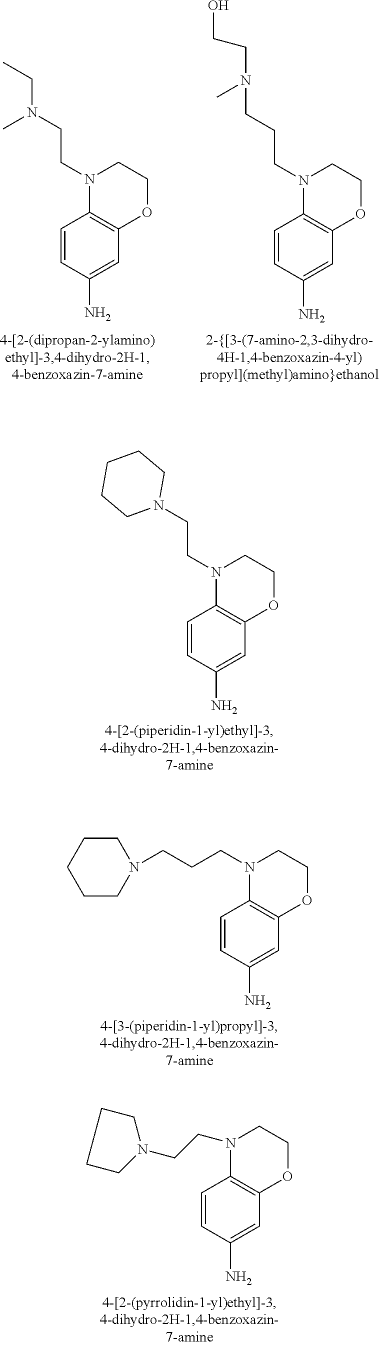 [SMILES: CCN(C)CCN1CCOc2cc(N)ccc21 ; CN(CCO)CCCN1CCOc2cc(N)ccc21 ; Nc1ccc2c(c1)OCCN2CCN1CCCCC1 ; Nc1ccc2c(c1)OCCN2CCCN1CCCCC1 ; Nc1ccc2c(c1)OCCN2CCN1CCCC1]

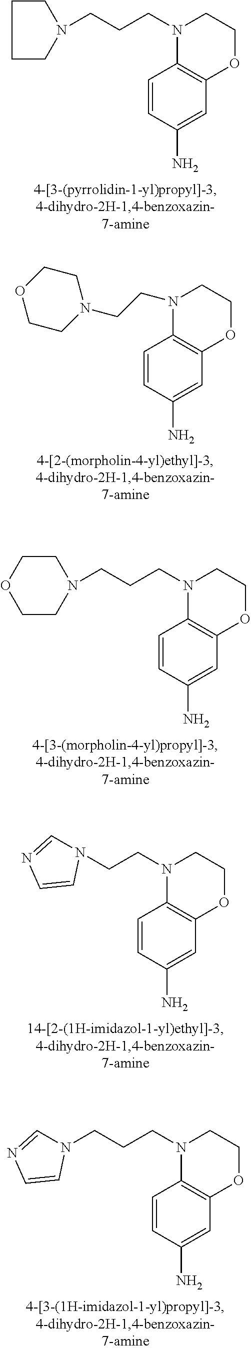 [SMILES: Nc1ccc2c(c1)OCCN2CCCN1CCCC1 ; Nc1ccc2c(c1)OCCN2CCN1CCOCC1 ; Nc1ccc2c(c1)OCCN2CCCN1CCOCC1 ; Nc1ccc2c(c1)OCCN2CCn1ccnc1 ; Nc1ccc2c(c1)OCCN2CCCn1ccnc1]

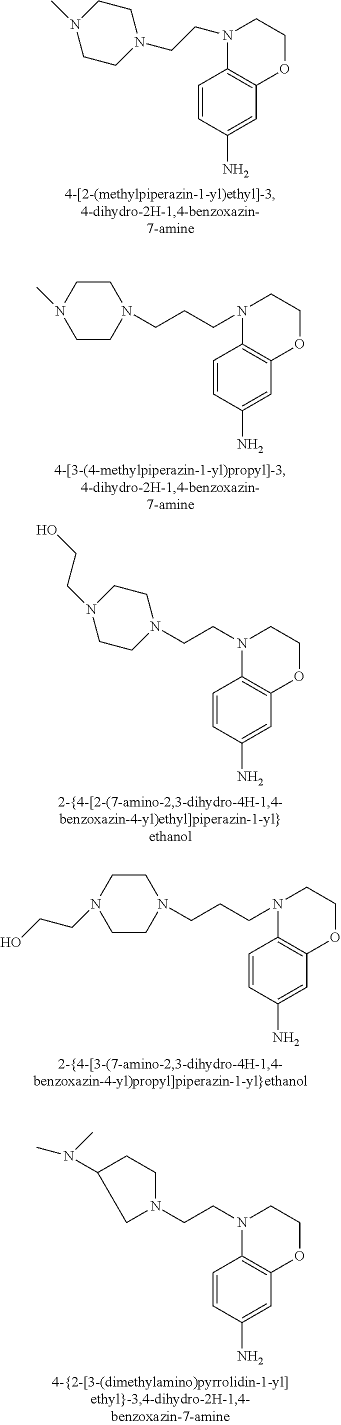 [SMILES: CN1CCN(CCN2CCOc3cc(N)ccc32)CC1 ; CN1CCN(CCCN2CCOc3cc(N)ccc32)CC1 ; Nc1ccc2c(c1)OCCN2CCN1CCN(CCO)CC1 ; Nc1ccc2c(c1)OCCN2CCCN1CCN(CCO)CC1 ; CN(C)C1CCN(CCN2CCOc3cc(N)ccc32)C1]

and also the addition salts, optical isomers, geometrical isomers, tautomers and/or solvates thereof.

### II/ According to a Second Embodiment of the Invention, the Compounds of Formula (I) are such That

R, R₂, R₃, R₄ and R₅ represent a hydrogen atom,

R₁ represents a linear C2-C10 or branched C3-C10 alkyl radical, interrupted with one or more non-adjacent oxygen atoms and/or with one or more non-adjacent groups —NR′, said alkyl radical ending with a group —NX₁X₂, preferably, R₁ represents a linear C2-C10 or branched C3-C10 alkyl radical, interrupted with a non-adjacent oxygen atom and/or with a non-adjacent group —NH, said alkyl radical ending with a group —NX₁X₂

X₁ and X₂ independently denote:
a hydrogen atom
a linear C1-C6 alkyl radical or a branched C3-C6 alkyl radical
a linear C1-C6 hydroxyalkyl radical or a branched C3-C6 hydroxyalkyl radical,

X₁ and X₂ may form, with the nitrogen atom that bears them, a saturated or unsaturated 5- to 8-membered heterocycle, in which one of the ring members may be a heteroatom chosen from O, S and N; said heterocycle possibly being substituted with one or more linear or branched C₁-C₄ alkyl, C₁-C₄ hydroxyalkyl or dimethylamino radicals.

R′ denotes a hydrogen atom or a linear C1-C4 or branched C3-C4 alkyl radical.

As examples of saturated or unsaturated 5- to 8-membered heterocycles in which one of the ring members may be a heteroatom chosen from O, S and N, mention may be made of imidazole, pyridine, piperazine, pyrrolidine, morpholine, pyrimidine, thiazole, benzimidazole, benzothiazole, oxazole, benzotriazole, triazole, benzoxazole and piperidine rings.

X₁ and X₂ may form, with the nitrogen atom that bears them, a heterocycle preferably chosen from imidazoles, piperazines, pyrrolidines, morpholines and piperidines, said heterocycle possibly being substituted with one or more linear or branched C₁-C₄ alkyl, C₁-C₄ hydroxyalkyl or dimethylamino radicals.

According to this second variant, the compounds of formula (I) that are particularly preferred are the following:

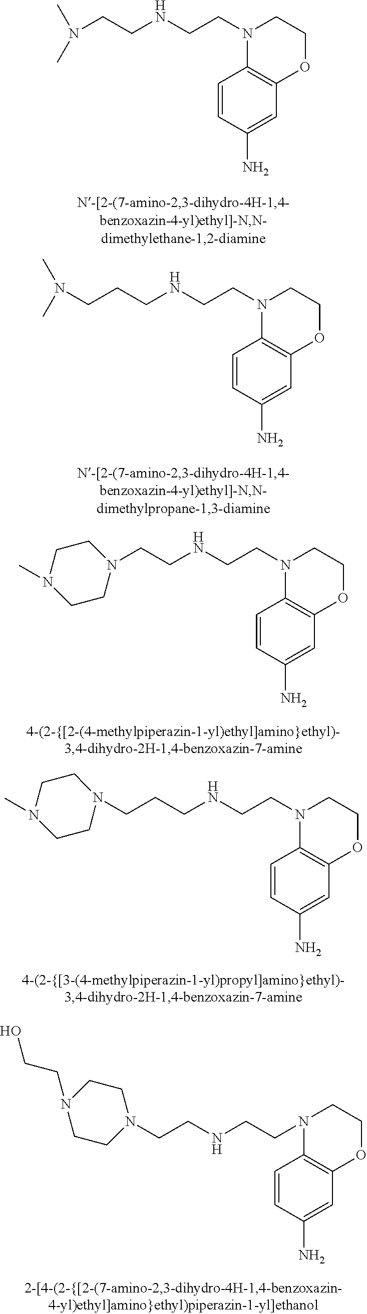 [SMILES: CN(C)CCNCCN1CCOc2cc(N)ccc21 ; CN(C)CCCNCCN1CCOc2cc(N)ccc21 ; CN1CCN(CCNCCN2CCOc3cc(N)ccc32)CC1 ; CN1CCN(CCCNCCN2CCOc3cc(N)ccc32)CC1 ; Nc1ccc2c(c1)OCCN2CCNCCN1CCN(CCO)CC1]

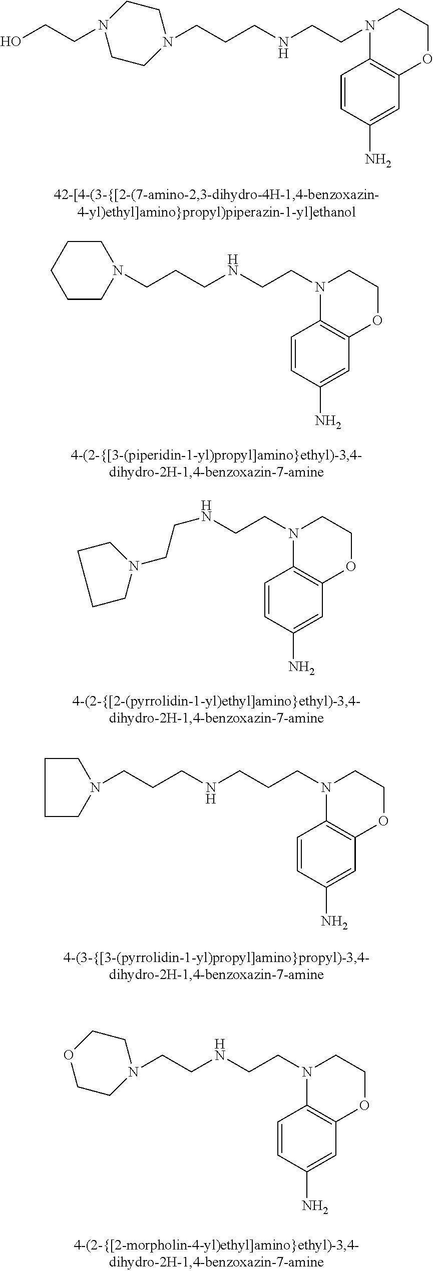 [SMILES: Nc1ccc2c(c1)OCCN2CCNCCCN1CCN(CCO)CC1 ; Nc1ccc2c(c1)OCCN2CCNCCCN1CCCCC1 ; Nc1ccc2c(c1)OCCN2CCNCCN1CCCC1 ; Nc1ccc2c(c1)OCCN2CCCNCCCN1CCCC1 ; Nc1ccc2c(c1)OCCN2CCNCCN1CCOCC1]

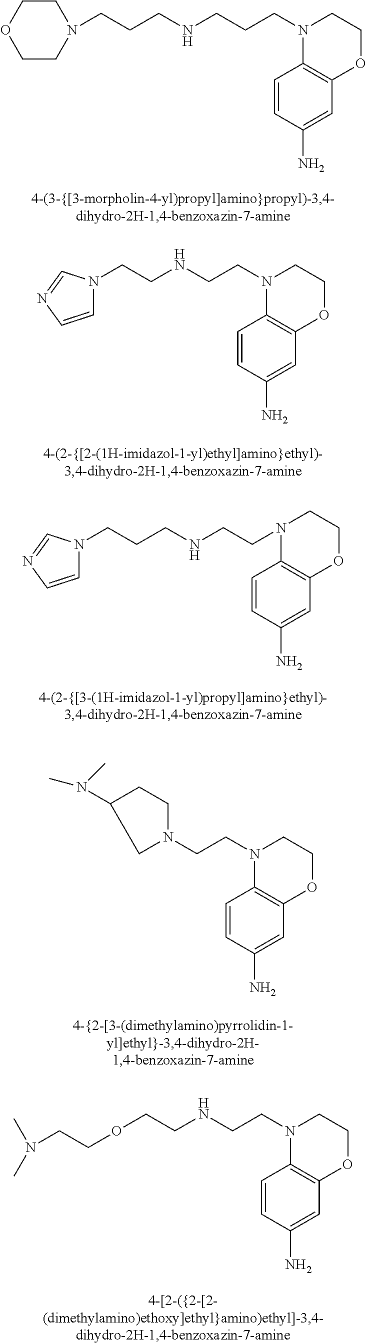 [SMILES: Nc1ccc2c(c1)OCCN2CCCNCCCN1CCOCC1 ; Nc1ccc2c(c1)OCCN2CCNCCn1ccnc1 ; Nc1ccc2c(c1)OCCN2CCNCCCn1ccnc1 ; CN(C)C1CCN(CCN2CCOc3cc(N)ccc32)C1 ; CN(C)CCOCCNCCN1CCOc2cc(N)ccc21]

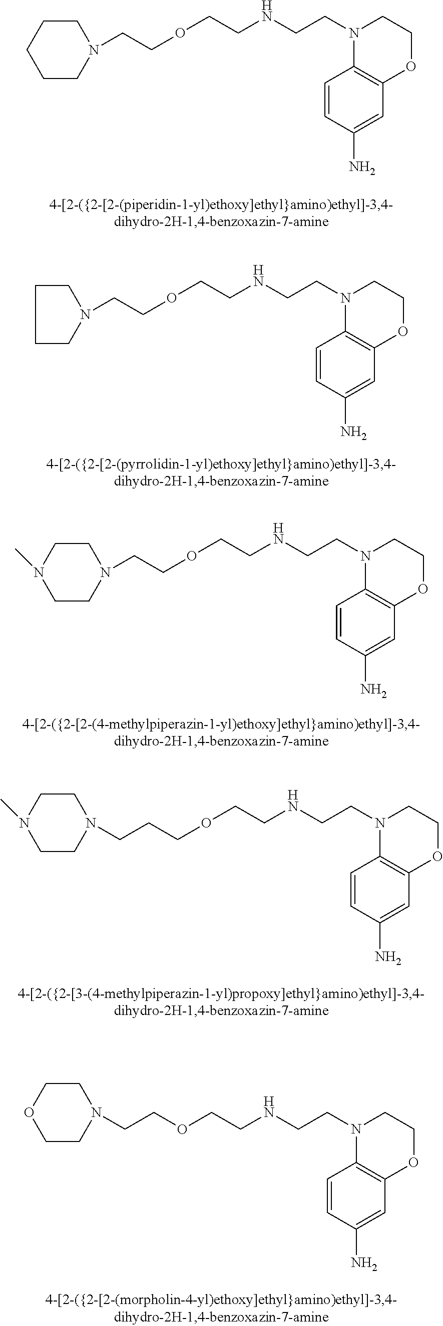 [SMILES: Nc1ccc2c(c1)OCCN2CCNCCOCCN1CCCCC1 ; Nc1ccc2c(c1)OCCN2CCNCCOCCN1CCCC1 ; CN1CCN(CCOCCNCCN2CCOc3cc(N)ccc32)CC1 ; CN1CCN(CCCOCCNCCN2CCOc3cc(N)ccc32)CC1 ; Nc1ccc2c(c1)OCCN2CCNCCOCCN1CCOCC1]

and also the addition salts, optical isomers, geometrical isomers, tautomers and/or solvates thereof.

### III/ According to a Third Embodiment of the Invention, the Compounds of Formula (I) are such That

R, R₂, R₃, R₄ and R₅ represent a hydrogen atom

R₁ represents a C2-C10 alkyl radical ending with a group —OX₃

X₃ denotes a hydrogen atom or a linear C1-C6 or branched C3-C6 alkyl radical, preferably a linear C1-C4 or branched C3-C4 alkyl radical. Preferably, X₃ denotes a hydrogen atom.

According to this embodiment, the compounds that are particularly preferred are chosen from:

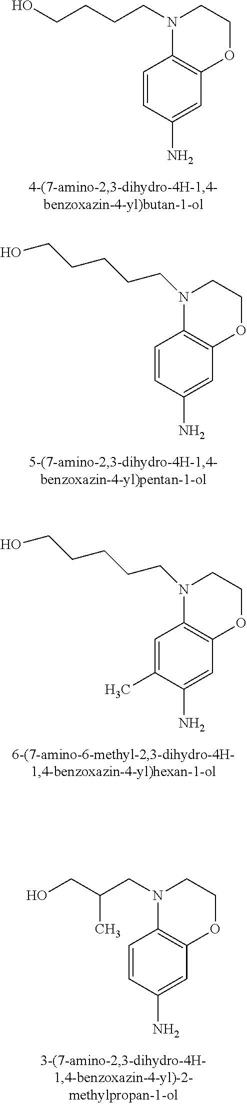

and also the addition salts, optical isomers, geometrical isomers, tautomers and/or solvates thereof.

### IV/ According to a Fourth Embodiment of the Invention, the Compounds of Formula (I) are such That

R, R₂, R₃, R₄ and R₅ represent a hydrogen atom

R₁ represents a linear C2-C10 or branched C3-C10 alkyl radical, interrupted with one or more oxygen atoms and/or with one or more groups —NR′, said alkyl radical ending with a group —OX₃,

R′ denotes a hydrogen atom or a linear C1-C4 or branched C3-C4 alkyl radical; preferably, R′ is a hydrogen atom,

X₃ denotes a hydrogen atom or a linear C1-C6 or branched C3-C6 alkyl radical, preferably a linear C1-C4 or branched C3-C4 alkyl radical or a hydrogen atom. Preferably, X₃ denotes a hydrogen atom.

According to this fourth variant, the compounds of formula (I) that are particularly preferred are the following:

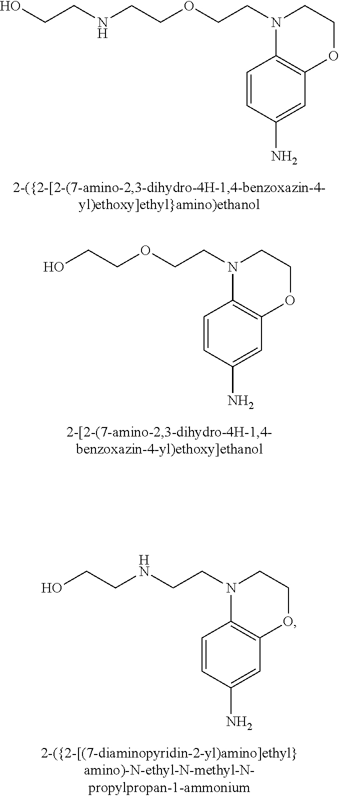 [SMILES: Nc1ccc2c(c1)OCCN2CCOCCNCCO ; Nc1ccc2c(c1)OCCN2CCOCCO ; Nc1ccc2c(c1)OCCN2CCNCCO]

and also the salts and/or solvates and optical isomers, geometrical isomers and tautomers thereof.

### V) According to a Fifth Preferred Variant of the Invention

R, R₂, R₃, R₄ and R₅ represent a hydrogen atom

R₁ represents a linear C2-C10 or branched C3-C10 alkyl radical, optionally interrupted with one or more non-adjacent oxygen atoms and/or with one or more non-adjacent groups —NR′, said alkyl radical ending with a radical chosen from:

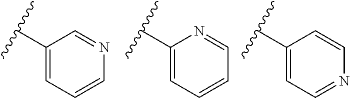

R′ denotes a hydrogen atom or a linear C1-C4 or branched C3-C4 alkyl radical; preferably, R′ is a hydrogen atom.

According to a particularly preferred embodiment, the compounds according to the invention are chosen from the compounds of general formula (I), and also the addition salts, optical isomers, geometrical isomers, tautomers and/or solvates thereof, in which:
R, R₂, R₃, R₄ and R₅ represent a hydrogen atom,
R₁ represents a linear C2-C10 or branched C3-C10 alkyl radical, preferably a linear C2-C6 or branched C3-C6 alkyl radical, optionally interrupted with a non-adjacent heteroatom chosen from O and S and/or with a non-adjacent group —NH, said alkyl radical ending with a group —NX₁X₂ or —OX₃,
X₁ and X₂ independently denote:
   a hydrogen atom
   a linear C1-C6 alkyl radical or a branched C3-C6 alkyl radical
   a linear C1-C6 hydroxyalkyl radical or a branched C3-C6 hydroxyalkyl radical,
X₁ and X₂ may form, with the nitrogen atom that bears them, a saturated or unsaturated 5- to 8-membered heterocycle, in which one of the ring members may be a heteroatom chosen from O, S and N; said heterocycle possibly being substituted with one or more linear or branched C₁-C₄ alkyl, C₁-C₄ hydroxyalkyl or dimethylamino radicals,
X₃ denotes a hydrogen atom or a linear C1-C4 or branched C3-C4 alkyl radical, preferably a hydrogen atom,

said saturated or unsaturated 5- to 8-membered heterocycle is chosen from imidazole, pyridine, piperazine, pyrrolidine, morpholine, pyrimidine, thiazole, benzimidazole, benzothiazole, oxazole, benzotriazole, triazole, pyrazole, benzoxazole and piperidine rings, preferably from imidazole, piperazine, pyrrolidine, morpholine and piperidine rings, preferably from imidazole, piperazine, pyrrolidine, morpholine and piperidine rings, which may be optionally substituted with one or more linear or branched C₁-C₄ alkyl, C₁-C₄ hydroxyalkyl or dimethylamino radicals.

A subject of the invention is also the use of a compound of formula (I′) below, addition salts thereof, optical isomers, geometrical isomers and tautomers thereof and/or solvates thereof:

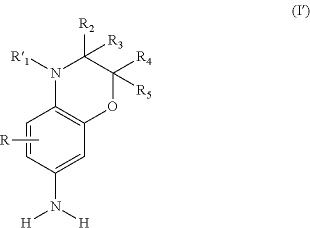

with:
R represents a hydrogen atom or a C1-C4 alkyl or C1-C4 hydroxyalkyl radical. Preferably, R represents a hydrogen atom,
R_{2,} R₃, R₄ and R₅ independently represent:
   a hydrogen atom,
   a linear C1-C4 or branched C3-C4 alkyl radical, optionally substituted with a hydroxyl radical,
R′₁ represents a radical X₄X₅N-Alk-,
Alk represents a linear or branched divalent C2-C10 alkyl radical, optionally interrupted with one or more non-adjacent heteroatoms chosen from O and S or with one or more non-adjacent groups —NR′ in which R′ denotes a hydrogen atom or a C1-C4 alkyl radical; preferably, Alk denotes a divalent C2-C5 alkyl radical optionally interrupted with an oxygen atom or with an —NH group,
   X₄ and X₅ independently denote:
      a hydrogen atom,
      a linear C1-C6 alkyl radical or a branched C3-C6 alkyl radical, optionally ending with a radical —OX₆, X₆ denoting a hydrogen atom or a linear C1-C6 or branched C3-C6 alkyl radical,
   X₄ and X₅ may form, with the nitrogen atom that bears them, a saturated or unsaturated 5- to 8-membered heterocycle, in which one of the ring members may be a heteroatom chosen from O, S and N; said heterocycle possibly being substituted with one or more linear or branched C₁-C₄ alkyl, C₁-C₄ hydroxyalkyl or dimethylamino radicals,
for dyeing keratin fibres, especially human keratin fibres such as the hair.

As examples of saturated or unsaturated 5- to 8-membered heterocycles in which one of the ring members may be a heteroatom chosen from O, S and N, mention may be made of imidazole, pyridine, piperazine, pyrrolidine, morpholine, pyrimidine, thiazole, benzimidazole, benzothiazole, oxazole, benzotriazole, triazole, pyrazole, benzoxazole and piperidine rings.

Preferably, said saturated or unsaturated 5- to 8-membered heterocycle in which one of the ring members may be a heteroatom chosen from O, S and N is chosen from imidazole, piperazine, pyrrolidine, morpholine and piperidine rings.

Preferably, R, R₂, R₃, R₄ and R₅ represent a hydrogen atom.

Preferably, R′₁ represents a radical X₄X₅N-Alk-, Alk representing a linear or branched C2-C5 divalent alkyl radical,
X₄ and X₅ independently denote a linear C1-C6 and preferably linear C2-C5 alkyl radical, optionally ending with a radical —OX₆, X₆ denoting a hydrogen atom or a linear C1-C6 and preferably linear C2-C5 alkyl radical, X₆ preferably denoting a hydrogen atom,
X₄ and X₅ may form, with the nitrogen atom that bears them, a saturated or unsaturated 5- to 8-membered heterocycle, in which one of the ring members may be a heteroatom chosen from O, S and N, chosen from imidazole, piperazine, pyrrolidine, morpholine and piperidine rings, said heterocycle possibly being substituted with one or more linear or branched C₁-C₄ alkyl, C₁-C₄ hydroxyalkyl or dimethylamino radicals.

Preferably:
X₄ and X₅ independently denote a linear C2-C5 alkyl radical or a linear C2-C5 alkyl radical ending with a radical —OX₆, X₆ denoting a linear C1-C6 and preferably linear C2-C5 alkyl radical or a hydrogen atom, X₆ preferably denoting a hydrogen atom, or
X₄ and X₅ form, with the nitrogen atom that bears them, an imidazole, piperazine, pyrrolidine, morpholine or piperidine heterocycle, said heterocycle possibly being substituted with one or more linear or branched C₁-C₄ alkyl, C₁-C₄ hydroxyalkyl or dimethylamino radicals.

Among the compounds of formula (I′), mention may be maid of the following:

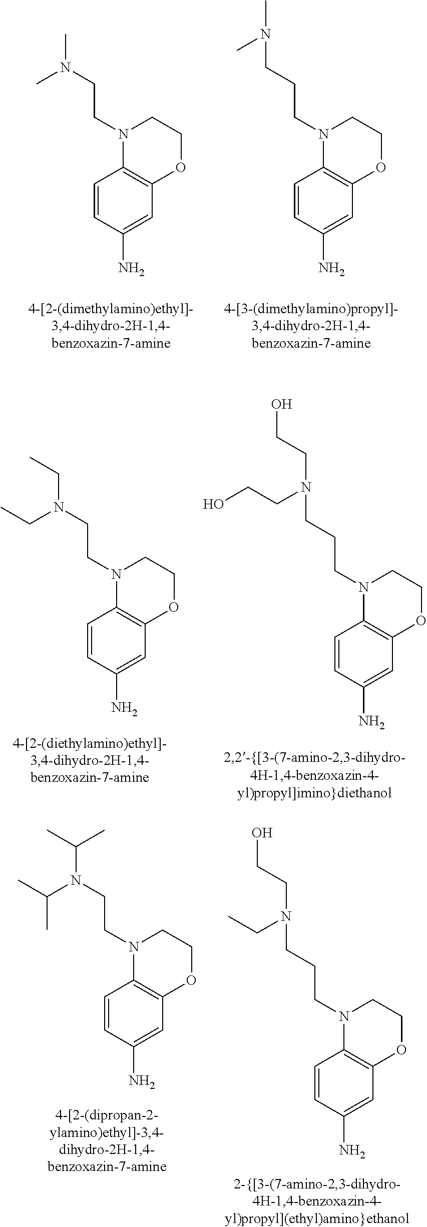 [SMILES: CN(C)CCN1CCOc2cc(N)ccc21 ; CN(C)CCCN1CCOc2cc(N)ccc21 ; CCN(CC)CCN1CCOc2cc(N)ccc21 ; Nc1ccc2c(c1)OCCN2CCCN(CCO)CCO ; CC(C)N(CCN1CCOc2cc(N)ccc21)C(C)C ; CCN(CCO)CCCN1CCOc2cc(N)ccc21]

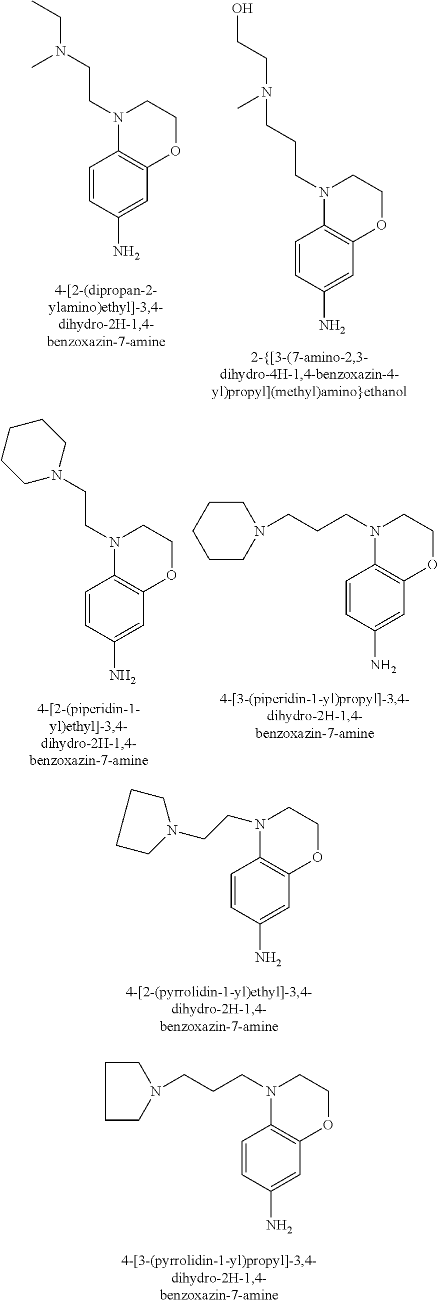

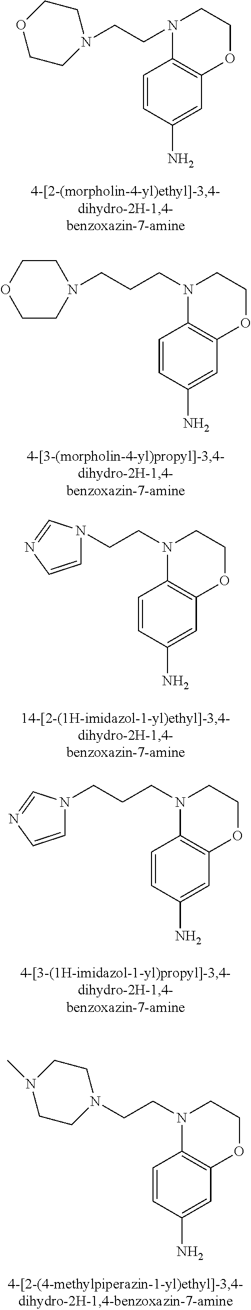 [SMILES: Nc1ccc2c(c1)OCCN2CCN1CCOCC1 ; Nc1ccc2c(c1)OCCN2CCCN1CCOCC1 ; Nc1ccc2c(c1)OCCN2CCn1ccnc1 ; Nc1ccc2c(c1)OCCN2CCCn1ccnc1 ; CN1CCN(CCN2CCOc3cc(N)ccc32)CC1]

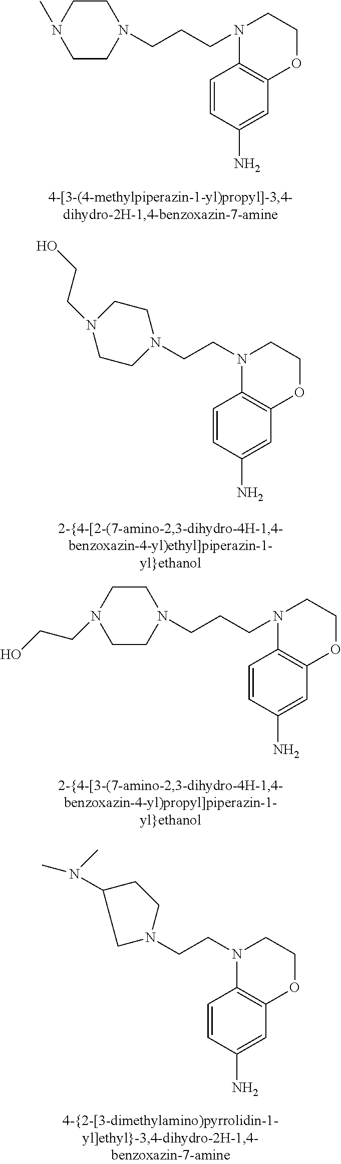 [SMILES: CN1CCN(CCCN2CCOc3cc(N)ccc32)CC1 ; Nc1ccc2c(c1)OCCN2CCN1CCN(CCO)CC1 ; Nc1ccc2c(c1)OCCN2CCCN1CCN(CCO)CC1 ; CN(C)C1CCN(CCN2CCOc3cc(N)ccc32)C1]

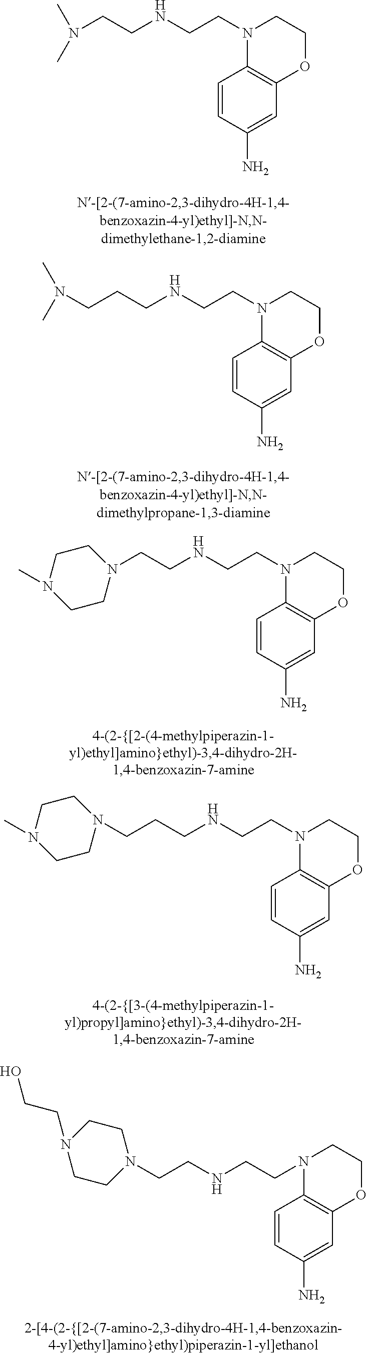 [SMILES: CN(C)CCNCCN1CCOc2cc(N)ccc21 ; CN(C)CCCNCCN1CCOc2cc(N)ccc21 ; CN1CCN(CCNCCN2CCOc3cc(N)ccc32)CC1 ; CN1CCN(CCCNCCN2CCOc3cc(N)ccc32)CC1 ; Nc1ccc2c(c1)OCCN2CCNCCN1CCN(CCO)CC1]

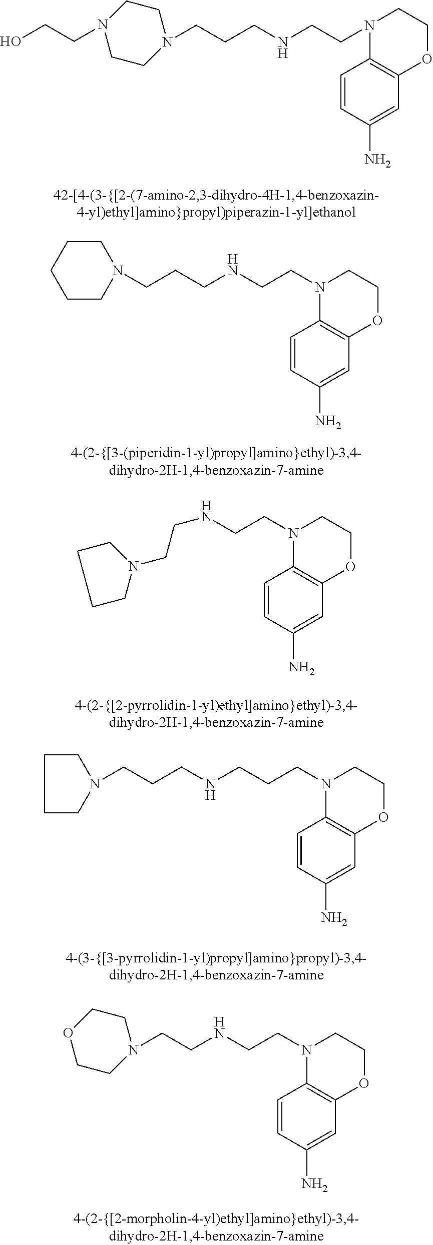 [SMILES: Nc1ccc2c(c1)OCCN2CCNCCCN1CCN(CCO)CC1 ; Nc1ccc2c(c1)OCCN2CCNCCCN1CCCCC1 ; Nc1ccc2c(c1)OCCN2CCNCCN1CCCC1 ; Nc1ccc2c(c1)OCCN2CCCNCCCN1CCCC1 ; Nc1ccc2c(c1)OCCN2CCNCCN1CCOCC1]

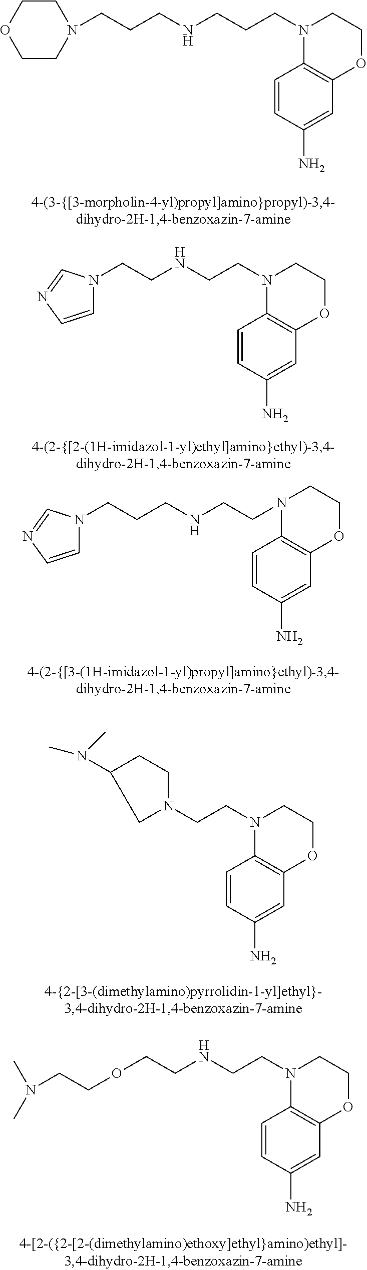 [SMILES: Nc1ccc2c(c1)OCCN2CCCNCCCN1CCOCC1 ; Nc1ccc2c(c1)OCCN2CCNCCn1ccnc1 ; Nc1ccc2c(c1)OCCN2CCNCCCn1ccnc1 ; CN(C)C1CCN(CCN2CCOc3cc(N)ccc32)C1 ; CN(C)CCOCCNCCN1CCOc2cc(N)ccc21]

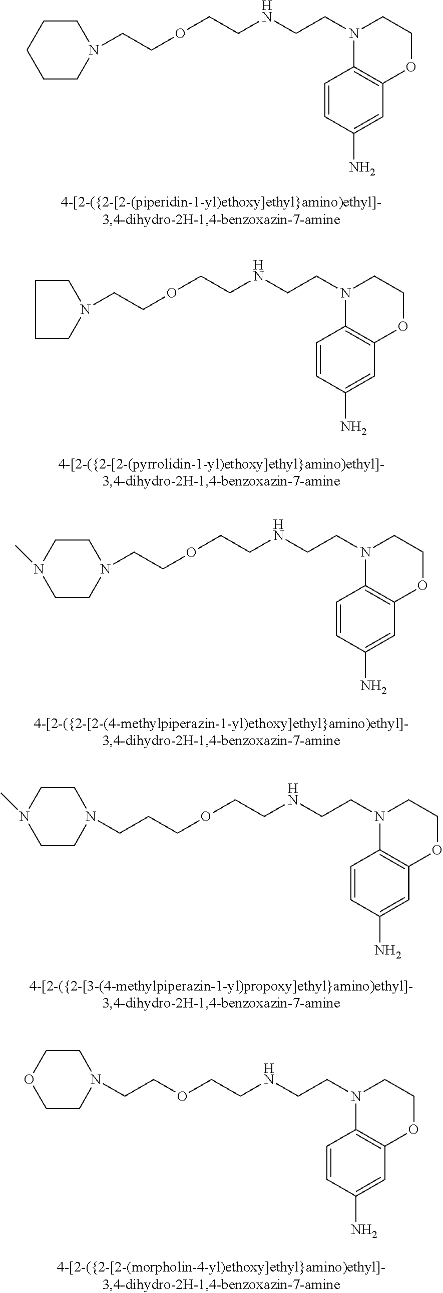 [SMILES: Nc1ccc2c(c1)OCCN2CCNCCOCCN1CCCCC1 ; Nc1ccc2c(c1)OCCN2CCNCCOCCN1CCCC1 ; CN1CCN(CCOCCNCCN2CCOc3cc(N)ccc32)CC1 ; CN1CCN(CCCOCCNCCN2CCOc3cc(N)ccc32)CC1 ; Nc1ccc2c(c1)OCCN2CCNCCOCCN1CCOCC1]

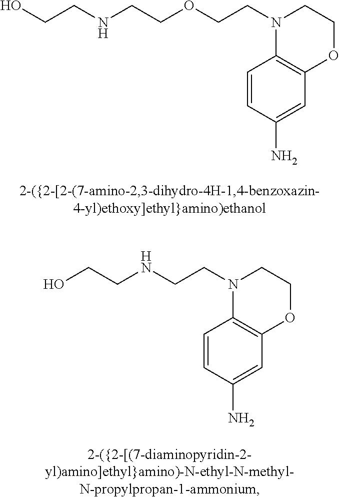 [SMILES: Nc1ccc2c(c1)OCCN2CCOCCNCCO ; Nc1ccc2c(c1)OCCN2CCNCCO]

and also the addition salts thereof, optical isomers, geometrical isomers and tautomers thereof and/or solvates thereof.

Benzoxazine-Based Compounds

A subject of the invention is also a compound of formula (II) below, addition salts thereof, optical isomers, geometrical isomers and tautomers thereof and/or solvates thereof:

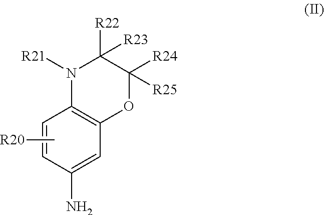

in which
R₂₁ represents a radical chosen from:
   a linear C4-C10 alkyl radical, preferably a linear C4-C6 alkyl radical, ending with a group —NX₂₁X₂₂,
   a branched C3-C10 alkyl radical, preferably a branched C3-C6 alkyl radical, ending with a group —NX₂₁X₂₂,
   a linear C2-C10 or branched C3-C10 alkyl radical, interrupted with one or more non-adjacent heteroatoms chosen from O and S or with one or more non-adjacent groups —NR′₂₀, said alkyl radical ending with a group —NX₂₁X₂₂ or —OX₂₃, preferably, a linear C2-C6 or branched C3-C6 alkyl radical, interrupted with an oxygen atom and/or with a group —NH, said alkyl radical ending with a group —NX₂₁X₂₂ or —OX₂₃,
   a linear C2-C3 alkyl radical ending with a group —NX₃₁X₃₂,
   a linear C2-C10 or branched C3-C10 alkyl radical, optionally interrupted with one or more non-adjacent heteroatoms chosen from O and S or with one or more non-adjacent groups —NR′₂₀, said alkyl radical ending with a radical chosen from

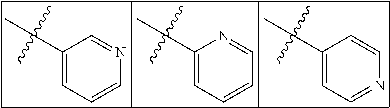

X₂₁ and X₂₂ independently denote:
a hydrogen atom
a linear C1-C6 alkyl radical or a branched C3-C6 alkyl radical
a linear C1-C6 hydroxyalkyl radical or a branched C3-C6 hydroxyalkyl radical,
X₂₁ and X₂₂ may form, with the nitrogen atom that bears them, a saturated or unsaturated 5- to 8-membered heterocycle, in which one of the ring members may be a heteroatom chosen from O, S and N; said heterocycle possibly being substituted with one or more linear or branched C₁-C₄ alkyl, C₁-C₄ hydroxyalkyl or dimethylamino radicals,
X₂₃ denotes a hydrogen atom or a linear C1-C6 or branched C3-C6 alkyl radical, preferably a linear C1-C4 or branched C3-C4 alkyl radical, X₂₃ preferably denoting a hydrogen atom,
R′₂₀ denotes a hydrogen atom or a linear C1-C4 or branched C3-C4 alkyl radical,
X₃₁ and X₃₂ independently denote:
a linear C2-C6 alkyl radical or a branched C3-C6 alkyl radical
a linear C1-C6 hydroxyalkyl radical or a branched C3-C6 hydroxyalkyl radical
X₃₃ denotes a linear C2-C6 or branched C3-C6 alkyl radical, preferably a linear C2-C4 or branched C3-C4 alkyl radical
R₂₀ represents a hydrogen atom or a C1-C4 alkyl or C1-C4 hydroxyalkyl radical. Preferably, R₂₀ represents a hydrogen atom,
R₂₂, R₂₃, R₂₄ and R₂₅ independently represent a hydrogen atom or a linear C1-C4 or branched C3-C4 alkyl radical, optionally substituted with a hydroxyl radical.

Preferably, R₂₀ represents a hydrogen atom.

Preferably, R₂₂, R₂₃, R₂₄ and R₂₅ represent a hydrogen atom or a linear C1-C4 or branched C3-C4 alkyl radical.

Preferably, R₂₂, R₂₃, R₂₄ and R₂₅ represent a hydrogen atom.

According to a preferred embodiment, in formula (II), R₂₁ represents a linear C2-C3 alkyl radical ending with a group —NX₃₁X₃₂,

X₃₁ and X₃₂ independently denote:
a linear C2-C6 alkyl radical or a branched C3-C6 alkyl radical
a linear C1-C6 hydroxyalkyl radical or a branched C3-C6 hydroxyalkyl radical.

As examples of saturated or unsaturated 5- to 8-membered heterocycles in which one of the ring members may be a heteroatom chosen from O, S and N, mention may be made of imidazole, pyridine, piperazine, pyrrolidine, morpholine, pyrimidine, thiazole, benzimidazole, benzothiazole, oxazole, benzotriazole, triazole, pyrazole, benzoxazole and piperidine rings.

Preferably, said saturated or unsaturated 5- to 8-membered heterocycle in which one of the ring members may be a heteroatom chosen from O, S and N is chosen from imidazole, piperazine, pyrrolidine, morpholine and piperidine rings.

As examples of compounds of formula (II), mention may be made of the following compounds:

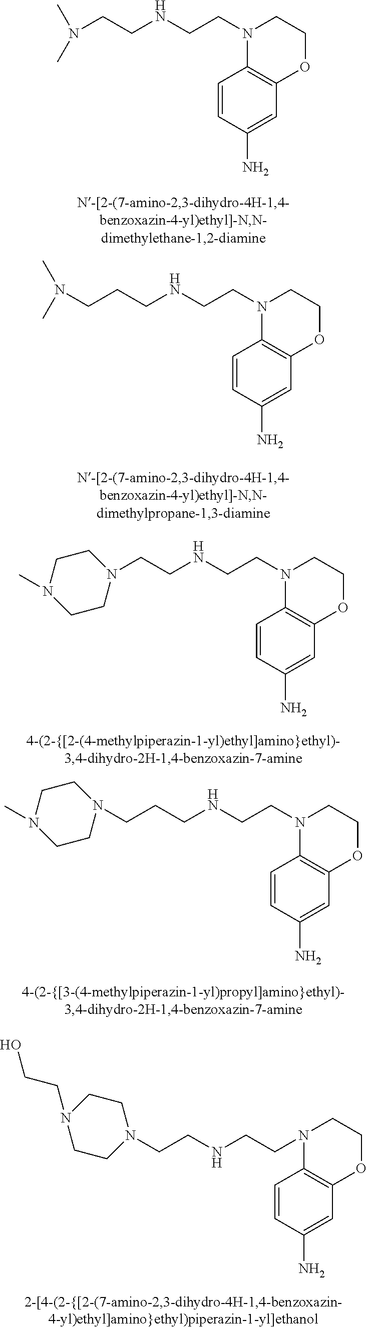 [SMILES: CN(C)CCNCCN1CCOc2cc(N)ccc21 ; CN(C)CCCNCCN1CCOc2cc(N)ccc21 ; CN1CCN(CCNCCN2CCOc3cc(N)ccc32)CC1 ; CN1CCN(CCCNCCN2CCOc3cc(N)ccc32)CC1 ; Nc1ccc2c(c1)OCCN2CCNCCN1CCN(CCO)CC1]

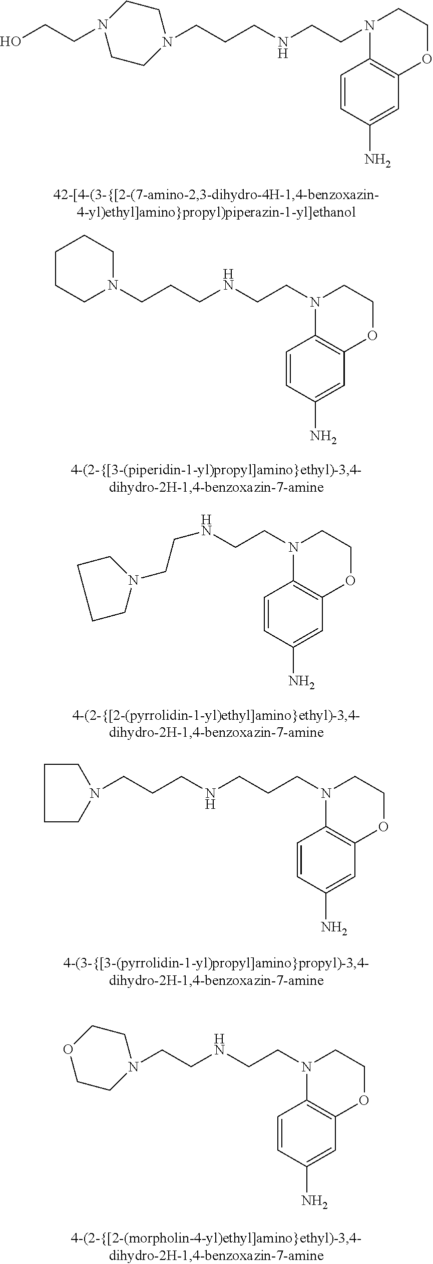 [SMILES: Nc1ccc2c(c1)OCCN2CCNCCCN1CCN(CCO)CC1 ; Nc1ccc2c(c1)OCCN2CCNCCCN1CCCCC1 ; Nc1ccc2c(c1)OCCN2CCNCCN1CCCC1 ; Nc1ccc2c(c1)OCCN2CCCNCCCN1CCCC1 ; Nc1ccc2c(c1)OCCN2CCNCCN1CCOCC1]

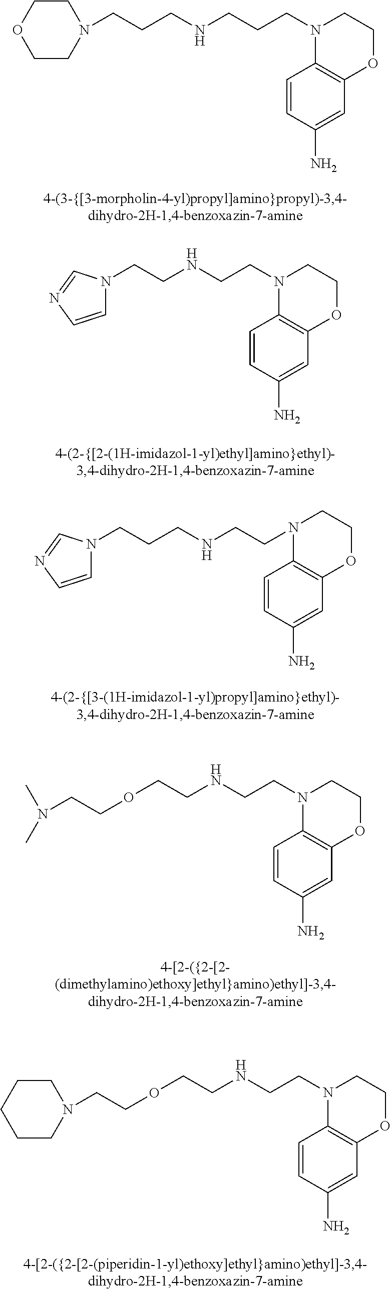 [SMILES: Nc1ccc2c(c1)OCCN2CCCNCCCN1CCOCC1 ; Nc1ccc2c(c1)OCCN2CCNCCn1ccnc1 ; Nc1ccc2c(c1)OCCN2CCNCCCn1ccnc1 ; CN(C)CCOCCNCCN1CCOc2cc(N)ccc21 ; Nc1ccc2c(c1)OCCN2CCNCCOCCN1CCCCC1]

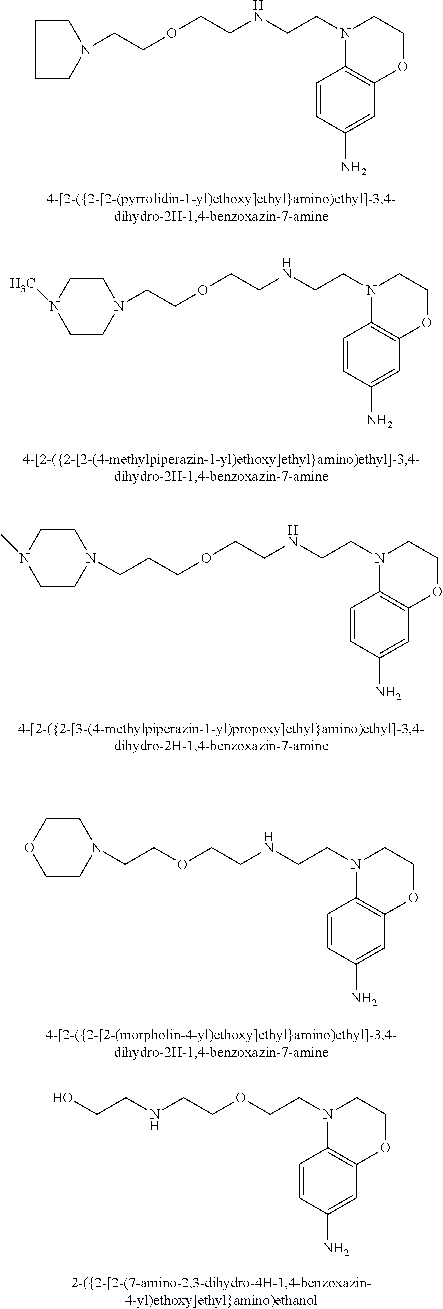 [SMILES: Nc1ccc2c(c1)OCCN2CCNCCOCCN1CCCC1 ; CN1CCN(CCOCCNCCN2CCOc3cc(N)ccc32)CC1 ; CN1CCN(CCCOCCNCCN2CCOc3cc(N)ccc32)CC1 ; Nc1ccc2c(c1)OCCN2CCNCCOCCN1CCOCC1 ; Nc1ccc2c(c1)OCCN2CCOCCNCCO]

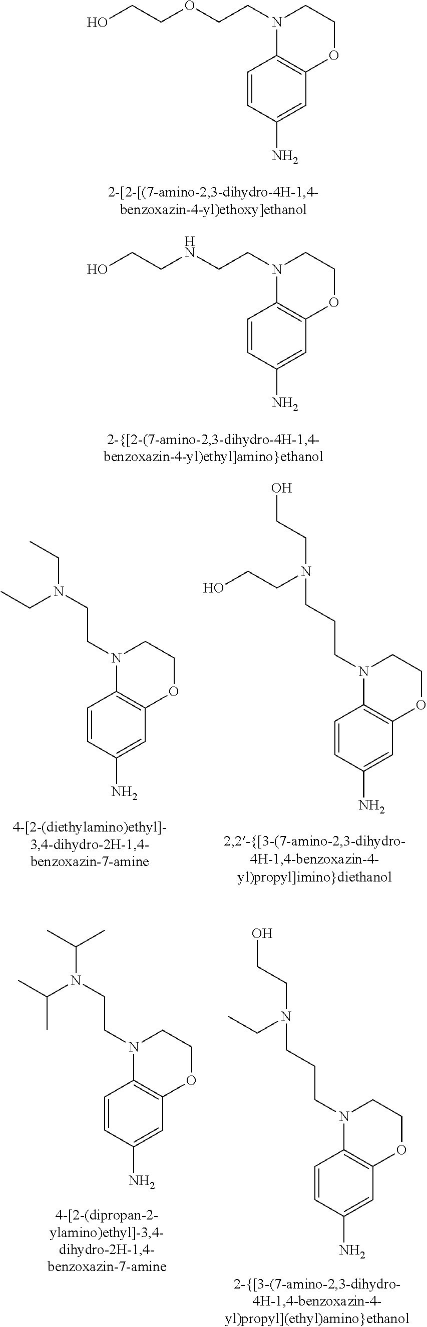 [SMILES: Nc1ccc2c(c1)OCCN2CCOCCO ; Nc1ccc2c(c1)OCCN2CCNCCO ; CCN(CC)CCN1CCOc2cc(N)ccc21 ; Nc1ccc2c(c1)OCCN2CCCN(CCO)CCO ; CC(C)N(CCN1CCOc2cc(N)ccc21)C(C)C ; CCN(CCO)CCCN1CCOc2cc(N)ccc21]

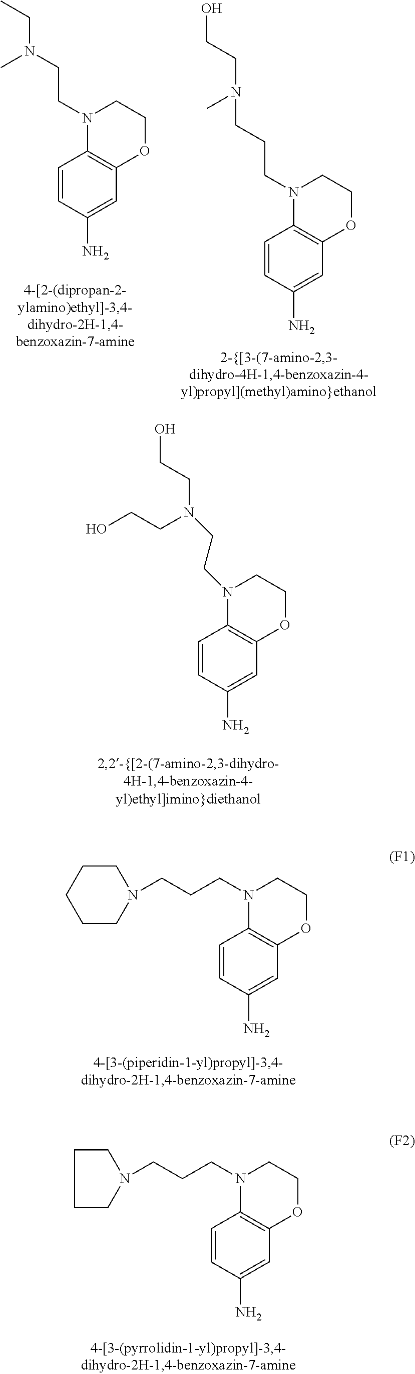 [SMILES: CCN(C)CCN1CCOc2cc(N)ccc21 ; CN(CCO)CCCN1CCOc2cc(N)ccc21 ; Nc1ccc2c(c1)OCCN2CCN(CCO)CCO ; Nc1ccc2c(c1)OCCN2CCCN1CCCCC1 ; Nc1ccc2c(c1)OCCN2CCCN1CCCC1]

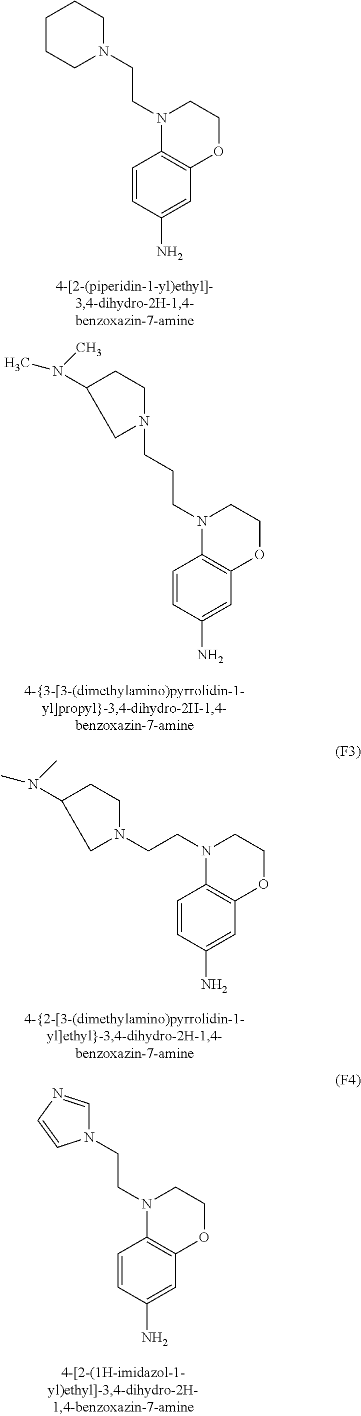 [SMILES: Nc1ccc2c(c1)OCCN2CCN1CCCCC1 ; CN(C)C1CCN(CCCN2CCOc3cc(N)ccc32)C1 ; CN(C)C1CCN(CCN2CCOc3cc(N)ccc32)C1 ; Nc1ccc2c(c1)OCCN2CCn1ccnc1]

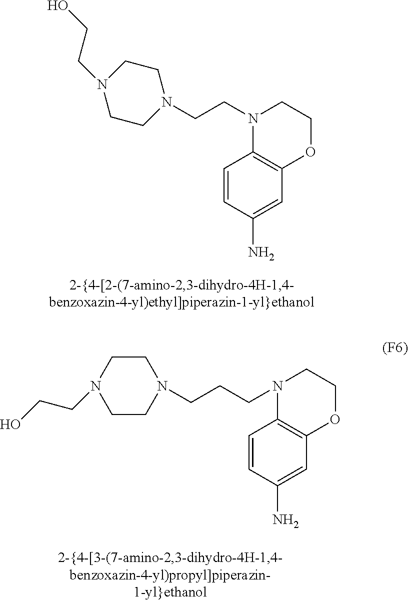 [SMILES: Nc1ccc2c(c1)OCCN2CCN1CCN(CCO)CC1 ; Nc1ccc2c(c1)OCCN2CCCN1CCN(CCO)CC1]

and also the salts and/or solvates, optical isomers, geometrical isomers and tautomers thereof.

A subject of the invention is also the following compounds:

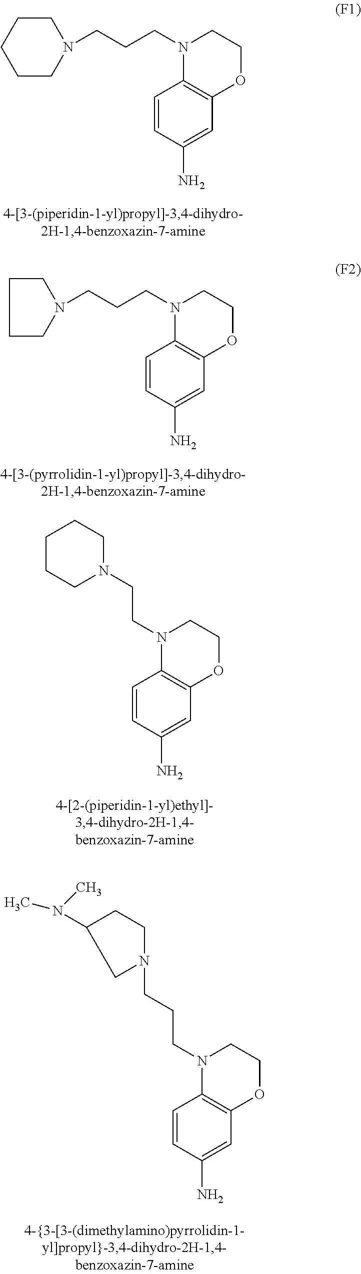 [SMILES: Nc1ccc2c(c1)OCCN2CCCN1CCCCC1 ; Nc1ccc2c(c1)OCCN2CCCN1CCCC1 ; Nc1ccc2c(c1)OCCN2CCN1CCCCC1 ; CN(C)C1CCN(CCCN2CCOc3cc(N)ccc32)C1]

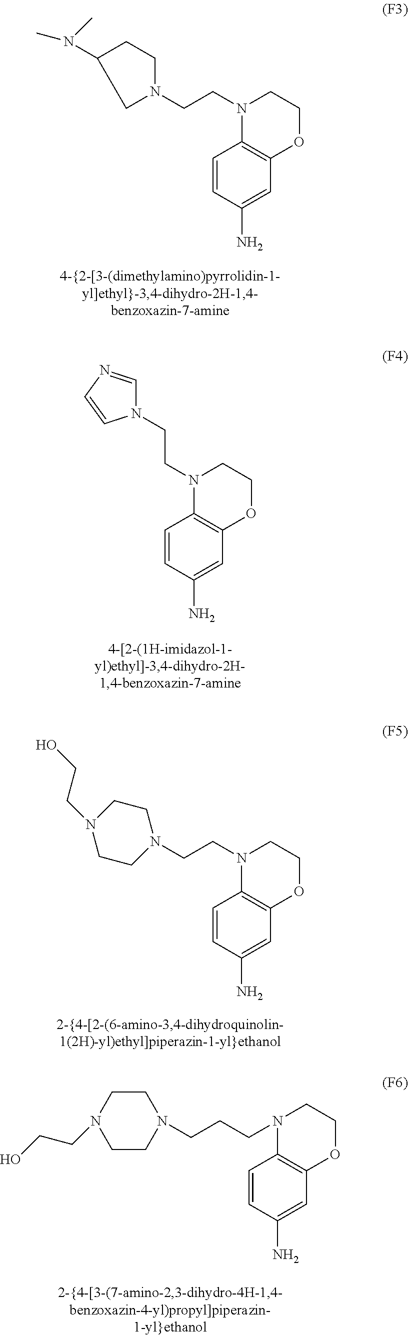 [SMILES: CN(C)C1CCN(CCN2CCOc3cc(N)ccc32)C1 ; Nc1ccc2c(c1)OCCN2CCn1ccnc1 ; Nc1ccc2c(c1)OCCN2CCN1CCN(CCO)CC1 ; Nc1ccc2c(c1)OCCN2CCCN1CCN(CCO)CC1]

and also the salts and/or solvates, optical isomers, geometrical isomers and tautomers thereof.

Dye Composition

The compounds of formulae (I), (I′), (II) and/or F1 to F6 as defined above may be used in a composition for dyeing keratin fibres comprising a suitable medium.

The compounds of formulae (I), (I′), (II) and/or F1 to F6 may each be present in the composition in an amount of between 0.001% and 10% and preferably between 0.005% and 6% by weight approximately relative to the total weight of the dye composition.

The composition may also comprise, besides said compounds of formulae (I), (I′), (II) and/or F1 to F6, at least one additional oxidation base. These bases may be chosen especially from para-phenylenediamines, bis(phenyl)alkylenediamines, para-aminophenols, ortho-aminophenols and heterocyclic bases, and the addition salts thereof.

Among the para-phenylenediamines, examples that may be mentioned more particularly include para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene, 3-hydroxy-1-(4′-aminophenyl)pyrrolidine, 6-(4-aminophenylamino)hexan-1-ol, N-(4-amino-3-methylphenyl)-N-[3-(1H-imidazol-1-yl)propyl]amine and N-(4-aminophenyl)-N-[3-(1H-imidazol-1-yl)propyl]amine, and the addition salts thereof with an acid.

Among the para-phenylenediamines mentioned above, para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine, 2-β-acetylaminoethyloxy-para-phenylenediamine and 2-[{2-[(4-aminophenyl)amino]ethyl}(2-hydroxyethyl)amino]ethanol, and the addition salts thereof with an acid, are particularly preferred.

Among the bis(phenyl)alkylenediamines, examples that may be mentioned include N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine and 1,8-bis(2,5-diaminophenoxy)-3,6-dioxaoctane, and the addition salts thereof with an acid.

Among the para-aminophenols, examples that may be mentioned include para-aminophenol, 4-amino-3-methylphenol, 4-amino-3-fluorophenol, 4-amino-2-chlorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, 4-amino-2-fluorophenol, 4-amino-2,6-dichlorophenol, 4-amino-6-[((5′-amino-2′-hydroxy-3′-methyl)phenyl)methyl]-2-methylphenol and bis[(5′-amino-2′-hydroxy)phenylmethane, and the addition salts thereof with an acid.

Among the ortho-aminophenols, examples that may be mentioned include 2-aminophenol, 2-amino-5-methylphenol, 2-amino-6-methylphenol and 5-acetamido-2-aminophenol, and the addition salts thereof with an acid.

Among the heterocyclic bases, examples that may be mentioned include pyridine derivatives, pyrimidine derivatives and pyrazole derivatives.

Among the pyridine derivatives, mention may be made of the compounds described, for example, in patents GB 1 026 978 and GB 1 153 196, such as 2,5-diaminopyridine, 2-(4-methoxyphenyl)amino-3-aminopyridine, 3,4-diaminopyridine, and the addition salts thereof with an acid.

Other pyridine oxidation bases that are useful in the present invention are the 3-aminopyrazolo[1,5-a]pyridine oxidation bases or the addition salts thereof, described, for example, in patent application FR 2 801 308. Examples that may be mentioned include pyrazolo[1,5-a]pyrid-3-ylamine, 2-acetylaminopyrazolo[1,5-a]pyrid-3-ylamine, 2-(morpholin-4-yl)pyrazolo[1,5-a]pyrid-3-ylamine, 3-aminopyrazolo[1,5-a]pyridine-2-carboxylic acid, 2-methoxypyrazolo[1,5-a]pyrid-3-ylamine, (3-aminopyrazolo[1,5-a]pyrid-7-yl)methanol, 2-(3-aminopyrazolo[1,5-a]pyrid-5-yl)ethanol, 2-(3-aminopyrazolo[1,5-a]pyrid-7-yl)ethanol, (3-aminopyrazolo[1,5-a]pyrid-2-yl)methanol, 3,6-diaminopyrazolo[1,5-a]pyridine, 3,4-diaminopyrazolo[1,5-a]pyridine, pyrazolo[1,5-a]pyridine-3,7-diamine, 7-(morpholin-4-yl)pyrazolo[1,5-a]pyrid-3-ylamine, pyrazolo[1,5-a]pyridine-3,5-diamine, 5-(morpholin-4-yl)pyrazolo[1,5-a]pyrid-3-ylamine, 2-[(3-aminopyrazolo[1,5-a]pyrid-5-yl)(2-hydroxyethyl)amino]ethanol, 2-[(3-aminopyrazolo[1,5-a]pyrid-7-yl)(2-hydroxyethyl)amino]ethanol, 3-aminopyrazolo[1,5-a]pyridin-5-ol, 3-aminopyrazolo[1,5-a]pyridin-4-ol, 3-aminopyrazolo[1,5-a]pyridin-6-ol and 3-aminopyrazolo[1,5-a]pyridin-7-ol, and addition salts thereof with an acid.

Among the pyridine bases that are of use in the present invention, mention may also be made of the compounds described in patent applications EP 1792903 and EP 1792606 and the addition salts thereof.

Among the pyrimidine derivatives, mention may be made of the compounds described, for example, in patents DE 2359399, JP 88-169571, JP 05-63124 and EP 0770375 or patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine and the addition salts thereof, and the tautomeric forms thereof, when a tautomeric equilibrium exists.

Among the pyrazolopyrimidine derivatives, mention may be made of the compounds described, for example, in patent applications EP 0847271, EP 0926149 and EP 1147109 and the addition salts thereof.

Among the pyrazole derivatives, mention may be made of the compounds described in patents DE 3843892, DE 4133957 and patent applications WO 94/08969, WO 94/08970, FR-A-2 733 749 and DE 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole, and the addition salts thereof.

As oxidation bases, mention may also be made of the diamino-N,N-dihydropyrazolone derivatives of formula (III) or an addition salt or solvate thereof:

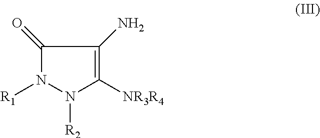

in which:

R₁, R₂, R₃ and R₄, which may be identical or different, represent:

a linear or branched C₁-C₆ alkyl radical optionally substituted with one or more radicals chosen from the group consisting of a radical OR₅, a radical NR₆R₇, a carboxyl radical, a sulfonic radical, a carboxamido radical CONR₆R₇, a sulfonamido radical SO₂NR₆R₇, a heteroaryl, an aryl optionally substituted with a (C₁-C₄)alkyl, hydroxyl, C₁-C₂ alkoxy, amino or (di)alkyl(C₁-C₂)amino group;

an aryl radical optionally substituted with one or more (C₁-C₄)alkyl, hydroxyl, C₁-C₂ alkoxy, amino or (di)alkyl(C₁-C₂)amino;

a 5- or 6-membered heteroaryl radical, optionally substituted with one or more radicals chosen from (C₁-C₄)alkyl and (C₁-C₂)alkoxy;

R₃ and R₄ may also represent a hydrogen atom;

R₅, R₆ and R₇, which may be identical or different, represent a hydrogen atom; a linear or branched C₁-C₄ alkyl radical optionally substituted with one or more radicals chosen from the group consisting of a hydroxyl, a C₁-C₂ alkoxy, a carboxamido CONR₈R₉, a sulfonyl SO₂R₈, an aryl optionally substituted with a (C₁-C₄)alkyl, a hydroxyl, a C₁-C₂ alkoxy, an amino or a (di)(C₁-C₂)alkylamino; an aryl optionally substituted with a (C₁-C₄)alkyl, a hydroxyl, a C₁-C₂ alkoxy, an amino or a (di)(C₁-C₂)alkylamino;

R₆ and R₇, which may be identical or different, may also represent a carboxamido radical CONR₈R₉; a sulfonyl radical SO₂R₈;

R₈ and R₉, which may be identical or different, represent a hydrogen atom; a linear or branched C₁-C₄ alkyl radical optionally substituted with one or more hydroxyl or C₁-C₂ alkoxy;

R₁ and R₂, on the one hand, and R₃ and R₄, on the other hand, may form, with the nitrogen atoms to which they are attached, a saturated or unsaturated 5- to 8-membered heterocycle optionally substituted with one or more radicals chosen from the group consisting of halogen atoms and amino, (di)alkyl(C₁-C₄)amino, hydroxyl, carboxyl, carboxamido and (C₁-C₂)alkoxy radicals, C₁-C₄ alkyl radicals optionally substituted with one or more hydroxyl, amino, (di)alkylamino, alkoxy, carboxyl or sulfonyl radicals;

R₃ and R₄ may also form, together with the nitrogen atom to which they are attached, a 5- or 7-membered heterocycle, the carbon atoms of which may be replaced with an optionally substituted oxygen or nitrogen atom.

These diamino-N,N-dihydropyrazolone derivatives are particularly described in patent application FR 2866338, a particularly preferred derivative being 2,3-diamino-6,7-dihydro-1H,5H-pyrazolo[1,2-a]pyrazol-1-one dimethanesulfonate.

Oxidation bases that may also be mentioned include the diamino-N,N-dihydropyrazolone derivatives of formula (IV) or an addition salt or solvate thereof:

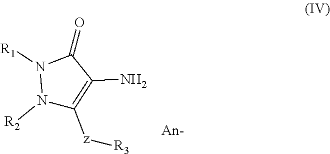

in which:

z represents independently:

a single covalent bond,

a divalent radical chosen from

an oxygen atom,

a radical —NR₆, with R₆ representing a hydrogen atom or a C₁-C₆ alkyl radical, or R₆, with R₃, forming, together with the nitrogen atom to which they are attached, a substituted or unsubstituted, saturated or unsaturated, aromatic or non-aromatic, 5- to 8-membered heterocycle, optionally containing one or more other heteroatoms or groups chosen from N, O, S, SO₂ and —CO—, it being possible for the heterocycle to be cationic and/or substituted with a cationic radical,

a radical —N⁺R₇R₈— with R₇ and R₈ independently representing an alkyl radical; the alkyl radical may be substituted with an OH or an —Oalkyl,

R₃ represents:

a hydrogen

a C₁-C₁₀ alkyl radical, which is optionally substituted, the alkyl radical possibly being interrupted with a heteroatom or a group chosen from O, N, Si, S, SO and SO₂,

a C₁-C₁₀ alkyl radical substituted and/or interrupted with a cationic radical,

a halogen,

an SO₃H radical,

a substituted or unsubstituted, saturated, unsaturated or aromatic, 5- to 8-membered ring, optionally containing one or more heteroatoms or groups chosen from N, O, S, SO₂ and —CO—, it being possible for the ring to be cationic and/or substituted with a cationic radical,

R₁ and R₂, which may be identical or different, represent:

a linear or branched C₁-C₆ alkyl radical optionally substituted with one or more radicals chosen from a radical OR₅, a radical NR₉R₁₀, a carboxyl radical, a sulfonic radical, a carboxamido radical CONR₉R₁₀, a sulfonamido radical SO₂NR₉R₁₀, a heteroaryl, an aryl optionally substituted with a (C₁-C₄)alkyl, hydroxyl, C₁-C₂ alkoxy, amino or (di)alkyl(C₁-C₂)amino group;

an aryl radical optionally substituted with one or more (C₁-C₄)alkyl, hydroxyl, C₁-C₂ alkoxy, amino or (di)alkyl(C₁-C₂)amino;

a 5- or 6-membered heteroaryl radical, optionally substituted with one or more radicals chosen from (C₁-C₄)alkyl monosubstituted or polysubstituted with an OH or an —Oalkyl or (C₁-C₂)alkoxy;

R₁ and R₂ may form, with the nitrogen atoms to which they are attached, a saturated or unsaturated 5- to 8-membered heterocycle optionally substituted with one or more radicals chosen from the group consisting of halogen atoms and amino, (di)alkyl(C₁-C₄)amino, hydroxyl, carboxyl, carboxamido and (C₁-C₂)alkoxy radicals, C₁-C₄ alkyl radicals optionally substituted with one or more hydroxyl, amino, (di)alkylamino, alkoxy, carboxyl or sulfonyl radicals;

An- represents an anion or a group of anions that ensures the electrical neutrality of the compounds of formula (IV),

on the condition that at least one of the groups Z and R₃ represents a cationic radical.

These derivatives of diamino-N,N-dihydropyrazolone are described in patent application FR 2 927 078.

In general, the concentration of the additional oxidation base(s) ranges from 0.0001% to 20% and preferably from 0.005% to 6% by weight relative to the total weight of the composition.

Couplers

The dye composition according to the invention may contain and preferably contains one or more additional oxidation couplers, other than the compounds of formulae (I), (I′) and (II), that are conventionally used for dyeing keratin fibres. Among these couplers, mention may be made especially of meta-phenylenediamines, meta-aminophenols, meta-diphenols, naphthalene couplers and heterocyclic couplers, and the addition salts thereof.

Examples of additional couplers that may be mentioned include 2-methyl-5-aminophenol, 5-N-(β-hydroxyethyl)amino-2-methylphenol, 6-chloro-2-methyl-5-aminophenol, 3-aminophenol, 2,4-dichloro-3-aminophenol, 5-amino-4-chloro-o-cresol, 1,3-dihydroxybenzene, 1,3-dihydroxy-2-methylbenzene, 4-chloro-1,3-dihydroxybenzene, 2,4-diamino-1-(8-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 1,5-dihydroxynaphthalene, 2,7-naphthalenediol, 1-acetoxy-2-methylnaphthalene, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 2,6-dihydroxy-3-4-dimethylpyridine, 3-amino-2-methylamino-6-methoxypyridine, 1-N-(β-hydroxyethyl)amino-3,4-methylenedioxybenzene, 2,6-bis(β-hydroxyethylamino)toluene and 3-methyl-1-phenyl-5-pyrazolone and the addition salts thereof with an acid.

In the dye composition of the present invention, the additional coupler(s), if they are present, generally represent an amount of between 0.001% and 10%, and preferably between 0.005% and 6% by weight approximately relative to the total weight of the composition.

The dye composition in accordance with the invention may also contain one or more direct dyes that may in particular be chosen from nitrobenzene dyes, azo direct dyes and methine direct dyes. These direct dyes may be of nonionic, anionic or cationic nature.

The medium that is suitable for dyeing, also known as the dye support, generally comprises water or a mixture of water and of one or more solvents, for instance C₁-C₄ lower alkanols such as ethanol and isopropanol, polyols, for instance propylene glycol, dipropylene glycol or glycerol, and polyol ethers, for instance dipropylene glycol monomethyl ether.

The solvent(s) are generally present in proportions that may be between 1% and 40% by weight approximately and more preferably between 3% and 30% by weight approximately relative to the total weight of the dye composition.

The dye composition in accordance with the invention may also contain various adjuvants conventionally used in hair dye compositions, such as anionic, cationic, nonionic, amphoteric or zwitterionic surfactants or mixtures thereof, anionic, cationic, nonionic, amphoteric or zwitterionic polymers or mixtures thereof, mineral or organic thickeners, and in particular anionic, cationic, nonionic and amphoteric polymeric associative thickeners, antioxidants, penetrants, sequestrants, fragrances, buffers, dispersants, conditioning agents, for instance volatile or non-volatile, modified or unmodified silicones, film-forming agents, ceramides, preserving agents and opacifiers.

The above adjuvants are generally present in an amount for each of them of between 0.01% and 20% by weight relative to the weight of the composition.

Needless to say, a person skilled in the art will take care to select this or these optional additional compound(s) such that the advantageous properties intrinsically associated with the oxidation dye composition in accordance with the invention are not, or are not substantially, adversely affected by the envisaged addition(s).

The pH of the dye composition in accordance with the invention is generally between 3 and 12 approximately and preferably between 5 and 11 approximately. It may be adjusted to the desired value by means of acidifying or basifying agents customarily used in the dyeing of keratin fibres, or alternatively using standard buffer systems.

Among the acidifying agents, examples that may be mentioned include mineral or organic acids, for instance hydrochloric acid, orthophosphoric acid, sulfuric acid, carboxylic acids, for instance acetic acid, tartaric acid, citric acid and lactic acid, and sulfonic acids.

Among the basifying agents, examples that may be mentioned include aqueous ammonia, alkali metal carbonates, alkanolamines such as monoethanolamine, diethanolamine and triethanolamine, and also derivatives thereof, sodium hydroxide, potassium hydroxide and the compounds of formula (III) below:

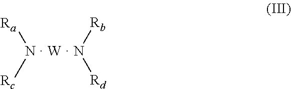

in which W is a propylene residue optionally substituted with a hydroxyl group or a C₁-C₄ alkyl radical; Rₐ, R_{b}, R_{c} and R_{d}, which may be identical or different, represent a hydrogen atom or a C₁-C₄ alkyl or C₁-C₄ hydroxyalkyl radical.

The composition according to the invention may comprise one or more oxidizing agents.

The oxidizing agents are those conventionally used for the oxidation dyeing of keratin fibres, for example hydrogen peroxide, urea peroxide, alkali metal bromates, persalts such as perborates and persulfates, peracids and oxidase enzymes, among which mention may be made of peroxidases, 2-electron oxidoreductases such as uricases, and 4-electron oxygenases, for instance laccases. Hydrogen peroxide is particularly preferred.

The dye composition with or without oxidizing agent according to the invention may be in various forms, such as in the form of liquids, creams or gels, or in any other form that is suitable for dyeing keratin fibres, and especially human hair.

It may result from the mixing, at the time of use, of several compositions.

In particular, it results from the mixing of at least two compositions, one comprising at least one compound of formulae (I), (I′), (II) and/or F1 to F6, optionally one or more oxidation bases, and optionally one or more additional couplers other than the compounds of formulae (I), (I′) and/or (II), or salts thereof, and a second composition comprising one or more oxidizing agents as described previously.

The composition of the invention is thus applied to the hair either in unmodified form or in the presence of one or more oxidizing agents, for the dyeing of keratin fibres.

The process of the present invention is a process in which the composition according to the present invention as defined previously is applied to the fibres, either alone or in the presence of an oxidizing agent, for a time that is sufficient to develop the desired colouring. The colour may be developed at acidic, neutral or alkaline pH, and the oxidizing agent may be added to the composition of the invention just at the time of use, or it may be used starting from an oxidizing composition which comprises it and which is applied simultaneously with or sequentially to the composition of the invention.

According to a particular embodiment, the composition is free of oxidizing agent and is mixed, preferably at the time of use, with a composition containing, in a medium that is suitable for dyeing, one or more oxidizing agents, these oxidizing agents being present in an amount sufficient to develop a colouring. The mixture obtained is then applied to the keratin fibres. After a leave-on time of approximately 3 to 50 minutes, preferably approximately 5 to 30 minutes, the keratin fibres are rinsed, washed with shampoo, rinsed again and then dried.

The oxidizing agents are those indicated above.

The oxidizing composition may also contain various adjuvants conventionally used in compositions for dyeing the hair and as defined above.

The pH of the oxidizing composition containing the oxidizing agent is such that, after mixing with the dye composition, the pH of the resulting composition applied to the keratin fibres preferably varies between 3 and 12 approximately and more preferably still between 5 and 11. It may be adjusted to the desired value by means of acidifying or basifying agents usually used in the dyeing of keratin fibres and as defined previously.

The ready-to-use composition that is finally applied to the keratin fibres may be in various forms, such as in the form of liquids, creams or gels or in any other form that is suitable for dyeing keratin fibres, and especially human hair.

A subject of the invention is also a dyeing “kit” or multi-compartment device in which a first compartment contains the dye composition devoid of oxidizing agent of the present invention defined above, comprising one or more oxidation bases chosen from the compound of formulae (I), (I′), (II) and/or F1 to F6 or the addition salts thereof with an acid, and a second compartment contains one or more oxidizing agents.

These devices may be equipped with a means for dispensing the desired mixture on the hair, such as the devices described in patent FR-2 586 913 in the name of the Applicant.

Preparation of the Compounds of Formulae (I), (I′), (II) and F1 to F6

The synthesis of the compounds of formulae (I), (I′), (II) and F1 to F6 may be performed, for example, according to the following procedures:

According to a particular embodiment, the synthesis of these compounds may be performed starting with the structural distributor “A” described in patent DE102008061864 and prepared according to the following scheme:

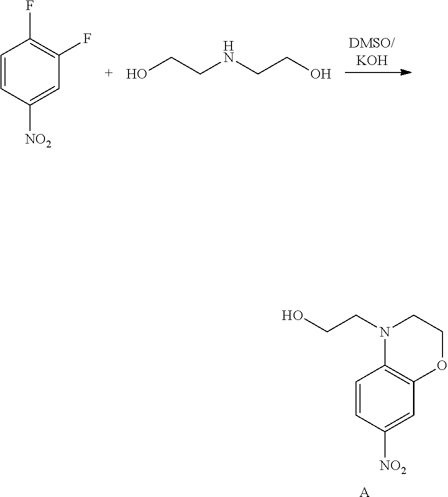

The structural distributor “A” is then used according to the reaction scheme below to give the compounds of structure (I).

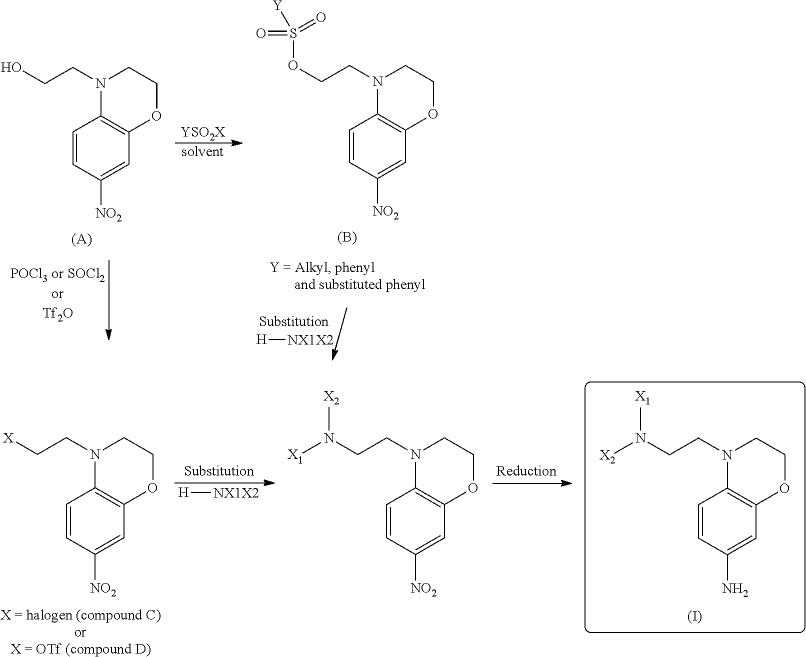

The compounds of formulae (I), (I′), (II) and F1 to F6 may also generally be prepared according to the following scheme:

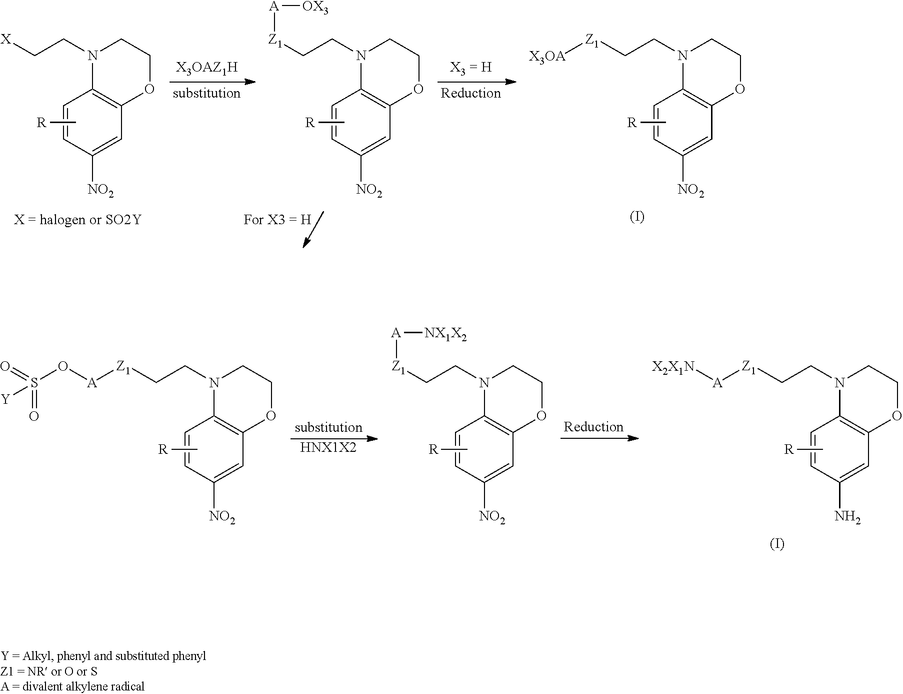

The substitution reaction is performed in a dipolar solvent such as acetonitrile, THF or in DMF or NMP, or in an alcohol such as ethanol for example, in the presence of a base such as triethylamine, ethyldiisopropylamine, sodium hydroxide or potassium hydroxide, for example, with one or more HZ₁AOX for 1 to 24 hours at a temperature from 20° C. to the reflux temperature of the solvent.

When X₃═H, the hydroxyl function thus introduced is then substituted with a halide (for example mesyl or tosyl halide) in a solvent such as acetonitrile or THF or in an alcohol such as ethanol, for example, in the presence of a base such as triethylamine, ethyldiisopropylamine, sodium hydroxide or potassium hydroxide, for example, for 1 to 24 hours at a temperature from 20° C. to the reflux temperature of the solvent.

The substitution of the leaving group introduced in the preceding step is performed either by reaction with an alcohol, an ether, a glycol derivative, an aromatic tertiary amine such as imidazole, or with a particular primary or secondary amine, for instance N,N-dimethylethylenediamine or 2-piperidin-1-ylethanamine, to give the expected compounds.

The reduction of the nitro group of these compounds is performed under standard conditions, for example by performing a hydrogenation reaction under heterogeneous catalysis in the presence of Pd/C, Pd(II)/C, Ni/Ra, etc., or alternatively by performing a reduction reaction with a metal, for example with zinc, iron, tin, etc. (see Advanced Organic Chemistry, 3rd Edition, J. March, 1985, Wiley Interscience and Reduction in Organic Chemistry, M. Hudlicky, 1983, Ellis Horwood Series Chemical Science).

The compounds of formulae (I), (I′), (II) and F1 to F6 may also be prepared according to the following scheme using the same reactions as those described previously:

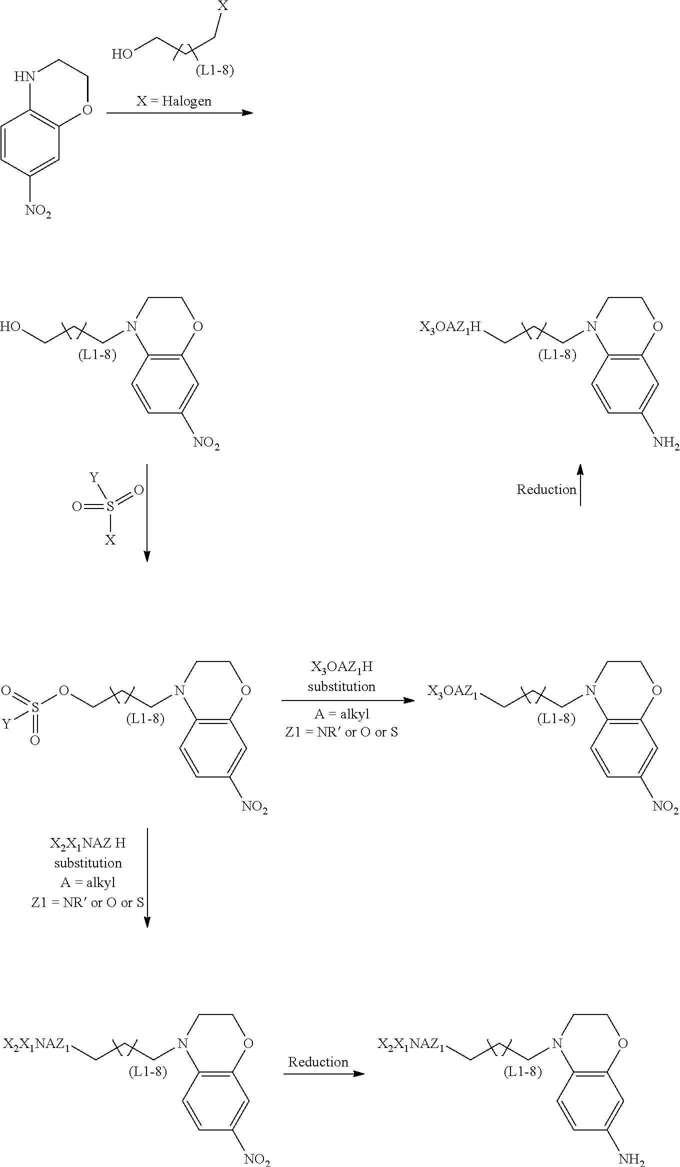

The examples that follow serve to illustrate the invention without, however, being limiting in nature.

### EXAMPLES OF SYNTHESIS

### Synthesis of the Structural Distributor B=2-(7-nitro-2,3-dihydro-4H-1,4-benzoxazin-4-yl)ethyl methanesulfonate

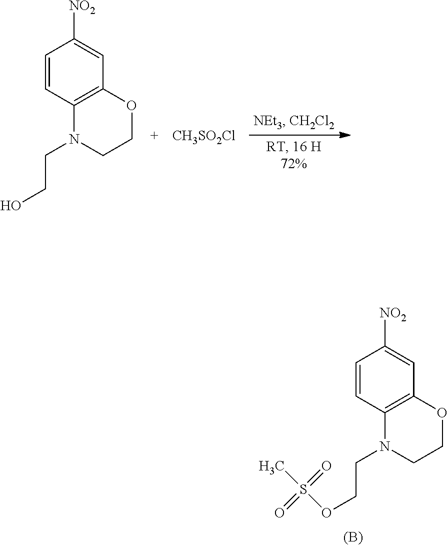

100 ml of dichloromethane, 6.7 g (0.030 mol) of 2-(7-nitro-2,3-dihydro-4H-1,4-benzoxazin-4-yl)ethanol and 6.8 g (0.06 mol) of mesyl chloride are successively placed in a 100 ml three-necked flask equipped with a thermometer, a condenser, a bubbler and a dropping funnel, with magnetic stirring. 6 g (0.060 mol) of triethylamine are added dropwise to this solution. This clear solution is stirred at room temperature for 16 hours.

The organic phase is washed with water and dried over anhydrous magnesium sulfate, and the solvent is evaporated off to give a crude product, which, after purification by recrystallization from ethyl acetate, gives 6.8 g (72% yield) of expected compound B in the form of a yellow powder.

The NMR (¹H 400 MHz and ¹³C 100.61 MHz DMSO-d₆) and mass spectrometry analyses are in accordance with the expected structure.

The mass spectrometry analysis confirms the expected compound C11H14N2O6. The quasi-molecular ions [M+H]+, [M+Na]+, [M−H]− of the expected molecule are mainly detected.

### Example 1

### Preparation of 4-[2-(diethylamino)ethyl]-3,4-dihydro-2H-1,4-benzoxazin-7-amine dihydrochloride hydrate

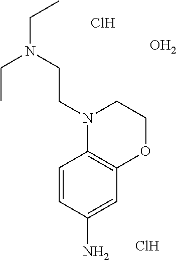 [SMILES: CCN(CC)CCN1CCOc2cc(N)ccc21 ; Cl ; Cl ; O]

### Synthesis of N,N-diethyl-2-(7-nitro-2,3-dihydro-4H-1,4-benzoxazin-4-yl)ethanamine

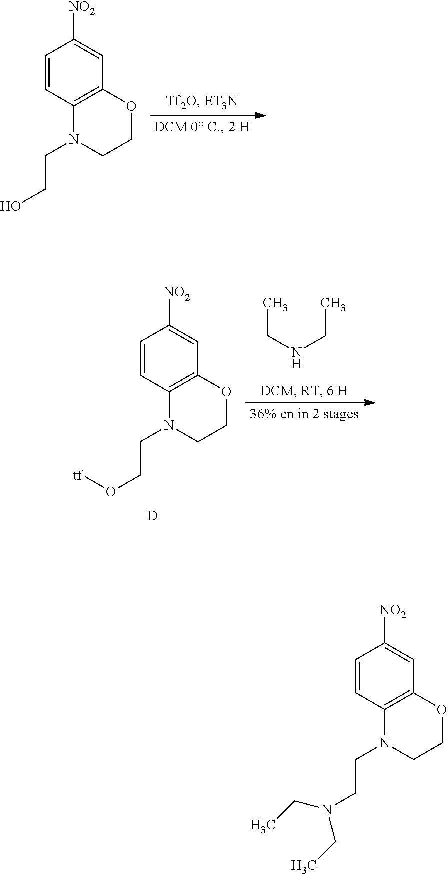

300 ml of dichloromethane, 8.9 g (0.080 mol) of structural distributor “A” 2-(7-nitro-2,3-dihydro-4H-1,4-benzoxazin-4-yl)ethanol and then 8 g (0.080 mol) of triethylamine are successively added at 0° C. to a 500 ml three-necked flask equipped with a thermometer, a condenser, a bubbler and a dropping funnel, with magnetic stirring. After stirring for 10 minutes, 14.6 g of Tf2O (52 mmol) are added. The medium is stirred for 2 hours at 0° C. and 5.6 g of diethylamine (80 mmol) are then added. The medium is then stirred for 6 hours at room temperature.

After evaporating off the solvent, the crude product obtained is purified by chromatography on a column of silica (CHCl2/MeOH=40/1) to give the expected product in the form of the trifluoroacetate salt.

The crude product is purified by recrystallization from an EtOAc/petroleum ether mixture (1/1). The product obtained is dissolved in 200 ml of saturated aqueous NaHCO₃ solution and extracted 3 times with dichloromethane. The combined organic phases are concentrated under vacuum to give 4 g of expected compound in the form of a brown oil (36% yield).

The NMR (¹H 400 MHz and ¹³C 100.61 MHz DMSO-d₆) and mass spectrometry analyses are in accordance with the expected structure.

Analysis by mass spectrometry confirms the expected compound C14H21N3O3. The quasi-molecular ions [M+H]+, [M+Na]+, [M−H]− of the expected molecule are mainly detected.

### Synthesis of 4-[2-(diethylamino)ethyl]-3,4-dihydro-2H-1,4-benzoxazin-7-amine dihydrochloride hydrate

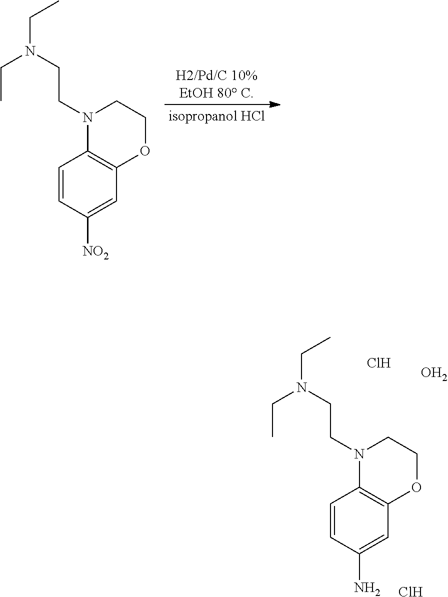 [SMILES: CCN(CC)CCN1CCOc2cc([N+](=O)[O-])ccc21 ; CCN(CC)CCN1CCOc2cc(N)ccc21 ; Cl ; O ; Cl]

This reduction is performed using an H-Cube hydrogenator containing a 90×4 mm cartridge of 10% Pd/C.

A solution of 1 g (0.0032 mol) of N,N-diethyl-2-(7-nitro-2,3-dihydro-4H-1,4-benzoxazin-4-yl)ethanamine in 300 ml of absolute ethanol is introduced at a flow rate of 3 ml per minute onto a cartridge of palladium catalyst at 80° C. under a pressure of 50 bar in the H-Cube system in the presence of hydrogen.

At the machine outlet, the pale beige solution obtained is added to a solution of 15 ml of 6N hydrochloric isopropanol. This solution is brought to 40° C. and the solvent is then removed by evaporation under vacuum.

The solid formed is recovered and dried under vacuum at 30° C. in the presence of desiccant, to give 1.4 g (100% yield) of expected compound in the form of a light-grey powder.

The NMR analyses (1D 1H and 2D 1H/13C NMR spectra of HMBC type) are in accordance with the expected structure.

The quasi-molecular ions [M+H]+, [M+Na]+ of the expected molecule C14H23N3O are mainly detected.

Elemental Analysis:

C(49.2); H(7.83); N(12.19); O(8.70); Cl(19.29)

### Example 2

### Preparation of 4-[2-(4-methylpiperazin-1-yl)ethyl]-3,4-dihydro-2H-1,4-benzoxazin-7-amine tetrahydrochloride

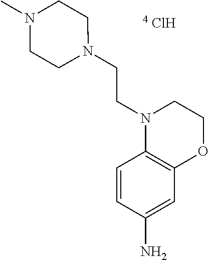 [SMILES: CN1CCN(CCN2CCOc3cc(N)ccc32)CC1 ; "4 ClH"]

### Synthesis of 4-[2-(4-methylpiperazin-1-yl)ethyl]-7-nitro-3,4-dihydro-2H-1,4-benzoxazine

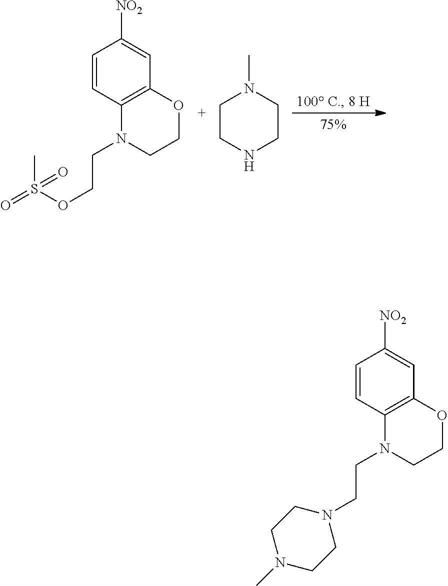

In a sealed tube, a mixture of 2 g (0.066 mol) of the compound 2-(7-nitro-2,3-dihydro-4H-1,4-benzoxazin-4-yl)ethyl methanesulfonate and 3 ml (0.027 mol) of N-methylpiperazine is maintained at 100° C. for 8 hours.

After cooling, the maximum amount of N-methylpiperazine is removed by evaporation under vacuum and the cooled crude product obtained is taken up in 100 ml of 1 N hydrochloric acid.

The pH of the solution obtained is adjusted to 8 and the solution is extracted several times with dichloromethane.

The combined organic phases are dried over anhydrous magnesium sulfate and then evaporated under vacuum on a rotavapor.

The crude product obtained is purified by chromatography on a column of silica (CH2Cl2/MeOH=40/1) to give 1.5 g (75% yield) of expected compound in the form of a brown-yellow solid.

The NMR (¹H 400 MHz and ¹³C 100.61 MHz DMSO-d₆) and mass spectrometry analyses are in accordance with the expected structure.

Analysis by mass spectrometry confirms the expected compound C15H22N4O3. The quasi-molecular ions [M+H]+, [M+Na]+, [M−H]− of the expected molecule are mainly detected.

### 4-[2-(4-Methylpiperazin-1-yl)ethyl]-3,4-dihydro-2H-1,4-benzoxazin-7-amine tetrahydrochloride

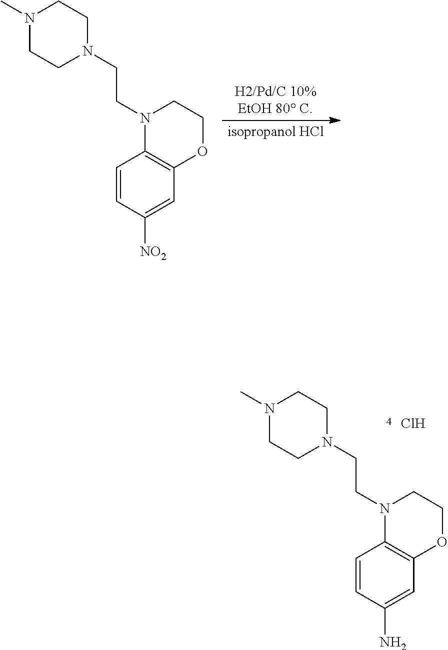 [SMILES: CN1CCN(CCN2CCOc3cc([N+](=O)[O-])ccc32)CC1 ; CN1CCN(CCN2CCOc3cc(N)ccc32)CC1 ; Cl]

This reduction is performed using an H-Cube hydrogenator containing a 90×4 mm cartridge of 10% Pd/C.

A solution of 1 g (0.0032 mol) of 4-[2-(4-methylpiperazin-1-yl)ethyl]-7-nitro-3,4-dihydro-2H-1,4-benzoxazine in 300 ml of absolute ethanol is introduced at a flow rate of 3 ml per minute onto a cartridge of palladium catalyst at 80° C. under a pressure of 50 bar in the H-Cube system in the presence of hydrogen.

At the machine outlet, the solution obtained is added to a solution of 15 ml of 6N hydrochloric isopropanol. This solution is brought to 40° C. and the solvent is then removed by evaporation under vacuum.

The solid formed is recovered and is dried under vacuum at 30° C. in the presence of desiccant, to give 1 g (72.6% yield) of compound in the form of a light-grey powder.

The NMR analyses (1D 1H and 2D 1H/13C NMR spectra of HMBC type) are in accordance with the expected structure.

The quasi-molecular ions [M+H]+, [M+Na]+ of the expected molecule are mainly detected.

Elemental Analysis:

C(37.2); H(6.43); N(11.31); O(10.47); Cl(31.04)

### Example 3

### Preparation of 2,2′-{[2-(7-amino-2,3-dihydro-4H-1,4-benzoxazin-4-yl)ethyl]imino}diethanol dihydrochloride

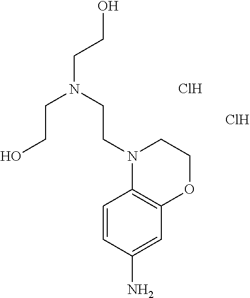 [SMILES: Nc1ccc2c(c1)OCCN2CCN(CCO)CCO ; Cl ; Cl]

### Synthesis of 2,2′-{[2-(7-nitro-2,3-dihydro-4H-1,4-benzoxazin-4-yl)ethyl]imino}diethanol

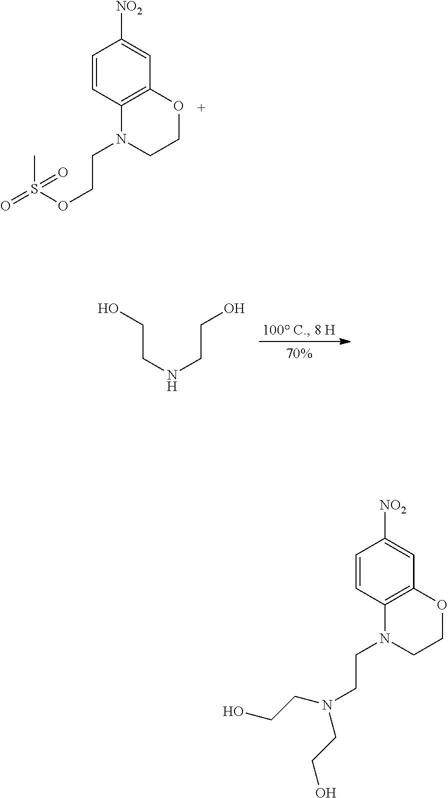 [SMILES: CS(=O)(=O)OCCN1CCOc2cc([N+](=O)[O-])ccc21 ; OCCNCCO ; O=[N+]([O-])c1ccc2c(c1)OCCN2CCN(CCO)CCO]

In a sealed tube, a mixture of 6.9 g (0.023 mol) of 2-(7-nitro-2,3-dihydro-4H-1,4-benzoxazin-4-yl)ethyl methanesulfonate and 10 ml (0.100 mol) of diethanolamine is maintained at 100° C. for 8 hours.

After cooling, the maximum amount of diethanolamine is removed by evaporation under vacuum and the cooled crude product obtained is taken up in 100 ml of 1 N hydrochloric acid.

The pH of the solution obtained is adjusted to 8 and the solution is extracted several times with dichloromethane.

The organic phase is dried over anhydrous magnesium sulfate and then evaporated under vacuum on a rotavapor.

The crude product obtained is purified by chromatography on a column of silica (CH2Cl2/MeOH=40/1) to give 5 g (70% yield) of expected compound in the form of a brown-yellow solid.

The NMR (1H 400 MHz and 13C 100.61 MHz DMSO-d₆) and mass spectrometry analyses are in accordance with the expected structure.

Analysis by mass spectrometry confirms the expected compound C14H21N3O5. The quasi-molecular ions [M+H]+, [M+Na]+, [M−H]− of the expected molecule are mainly detected.

### Synthesis of 2,2′-{[2-(7-amino-2,3-dihydro-4H-1,4-benzoxazin-4-yl)ethyl]imino}diethanol dihydrochloride

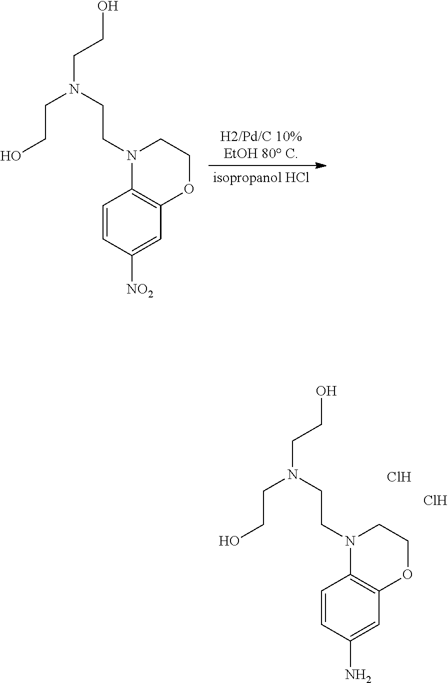 [SMILES: O=[N+]([O-])c1ccc2c(c1)OCCN2CCN(CCO)CCO ; Nc1ccc2c(c1)OCCN2CCN(CCO)CCO ; Cl ; Cl]

This reduction is performed using an H-Cube hydrogenator containing a 90×4 mm cartridge of 10% Pd/C.

A solution of 1 g (0.0032 mol) of 2,2′-{[2-(7-nitro-2,3-dihydro-4H-1,4-benzoxazin-4-yl)ethyl]imino}diethanol in 300 ml of absolute ethanol is introduced at a flow rate of 3 ml per minute onto a cartridge of palladium catalyst at 80° C. under a pressure of 50 bar in the H-Cube system in the presence of hydrogen.

At the machine outlet, the solution obtained is added to a solution of 15 ml of 6N hydrochloric isopropanol. This solution is brought to 40° C. and the solvent is then removed by evaporation under vacuum.

The solid formed is recovered and dried under vacuum at 30° C. in the presence of desiccant, to give 0.88 g (77.2% yield) of expected compound in the form of a light-grey powder.

The NMR analyses (1D 1H and 2D 1H/13C NMR spectra of HMBC type) are in accordance with the expected structure.

The quasi-molecular ions [M+H]+, [M+Na]+, [M+Cl]− of the expected molecule are mainly detected.

Elemental Analysis:

C(46.70); H(7.07); N(11.601); O(13.36); Cl(20.30)

### EXAMPLES OF DYE COMPOSITIONS

The following dye compositions were prepared:

| Example | 1 | | | | |
|---|---|---|---|---|---|
| 4-[2-(Diethylamino)ethyl]-3,4- | 10⁻³ mol | 10⁻³ mol | 10⁻³ mol | 10⁻³ mol | 10⁻³ mol |
| dihydro-2H-1,4-benzoxazin-7- | | | | | |
| amine hydrochloride hydrate | | | | | |
| 2-(2,4-Diaminophenoxy)ethanol | 10⁻³ mol | | | | |
| hydrochloride | | | | | |
| 5-Amino-2-methylphenol | | 10⁻³ mol | | | |
| 2-Methyl-5- | | | 10⁻³ mol | | |
| hydroxyethylaminophenol | | | | | |
| 6-Hydroxybenzomorpholine | | | | 10⁻³ mol | |
| 2-Amino-3-hydroxypyridine | | | | | 10⁻³ mol |
| Dye support (1) | (*) | (*) | (*) | (*) | (*) |
| Demineralized water qs | 100 g | 100 g | 100 g | 100 g | 100 g |
| Shade observed | Strong | Ash | Neutral | Matt | Neutral |
| | chromatic | violet | violet | yellow | grey |
| | blue | | | | |
| Example | 2 | | | | |
| 4-[2-(4-Methylpiperazin-1- | 10⁻³ mol | 10⁻³ mol | 10⁻³ mol | 10⁻³ mol | 10⁻³ mol |
| yl)ethyl]-3,4-dihydro-2H-1,4- | | | | | |
| benzoxazin-7-amine | | | | | |
| tetrahydrochloride | | | | | |
| 2-(2,4-Diaminophenoxy)ethanol | 10⁻³ mol | | | | |
| hydrochloride | | | | | |
| 5-Amino-2-methylphenol | | 10⁻³ mol | | | |
| 2-Methyl-5- | | | 10⁻³ mol | | |
| hydroxyethylaminophenol | | | | | |
| 6-Hydroxybenzomorpholine | | | | 10⁻³ mol | |
| 2-Amino-3-hydroxypyridine | | | | | 10⁻³ mol |
| Dye support (1) | (*) | (*) | (*) | (*) | (*) |
| Demineralized water qs | 100 g | 100 g | 100 g | 100 g | 100 g |
| Shade observed | Strong | Ash | Neutral | Grey | Light |
| | chromatic | violet- | violet | | gold |
| | blue-green | grey | | | |
| Example | 3 | | | | |
| 2,2′-{[2-(7-Amino-2,3-dihydro- | 10 mol | 10⁻³ mol | 10⁻³ mol | 10⁻³ mol | 10⁻³ mol |
| 4H-1,4-benzoxazin-4-yl) | | | | | |
| ethyl]imino}diethanol | | | | | |
| dihydrochloride | | | | | |
| 2-(2,4-Diaminophenoxy)ethanol | 10⁻³ mol | | | | |
| hydrochloride | | | | | |
| 5-Amino-2-methylphenol | | 10⁻³ mol | | | |
| 2-Methyl-5- | | | 10⁻³ mol | | |
| hydroxyethylaminophenol | | | | | |
| 6-Hydroxybenzomorpholine | | | | 10⁻³ mol | |
| 2-Amino-3-hydroxypyridine | | | | | 10⁻³ mol |
| Dye support (1) | (*) | (*) | (*) | (*) | (*) |
| Demineralized water qs | 100 g | 100 g | 100 g | 100 g | 100 g |
| Shade observed | Strong | Ash | Neutral | Grey | Ash |
| | chromatic | violet- | violet- | | grey |
| | blue | grey | grey | | |

(*): dye support (1) pH 9.5

| | |
|---|---|
| 96° ethyl alcohol | 20.8 g |
| 35% aqueous sodium metabisulfite solution | 0.23 g AM |
| Pentasodium salt of diethylenetriaminepentaacetic acid as an aqueous | 0.48 g AM |
| 40% solution | |
| C8-C10 alkyl polyglucoside as an aqueous 60% solution | 3.6 g AM |
| Benzyl alcohol | 2.0 g |
| Polyethylene glycol containing 8 units of ethylene oxide | 3.0 g |
| NH₄Cl | 4.32 g |
| Aqueous ammonia containing 20% NH₃ | 2.94 g |

At the time of use, each composition is mixed with an equal weight of 20-volumes aqueous hydrogen peroxide solution (6% by weight). A final pH of 9.5 is obtained.

Each mixture obtained is applied to locks of grey hair containing 90% white hairs. After a leave-in time of 30 minutes, the locks are rinsed, washed with a standard shampoo, rinsed again and then dried, to give the shades mentioned.

1.-14. (canceled)

15. A method for dyeing keratin fibers, comprising applying to the fibers a composition, wherein the composition comprises at least one compound corresponding to formula (I′) below, addition salts thereof, optical isomers, geometrical isomers and tautomers thereof, or solvates thereof: 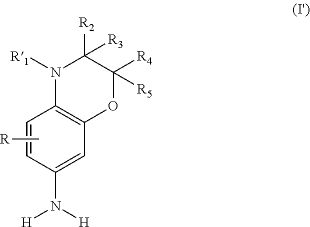
wherein:
R is chosen from a hydrogen atom, a C₁-C₄ alkyl, or a C₁-C₄ hydroxyalkyl radical,
R₂, R₃, R₄ and R₅, independently of each other, are chosen from a hydrogen atom, or a linear C₁-C₄ or branched C₃-C₄ alkyl radical, optionally substituted with a hydroxyl radical,
R′₁ is chosen from a radical X₄X₅N-Alk-,
wherein:
Alk is chosen from a linear or branched divalent C₂-C₁₀ alkyl radical, optionally interrupted with at least one non-adjacent heteroatom chosen from O, S, or at least one non-adjacent group —NR′ wherein R′ is chosen from a hydrogen atom or a C₁-C₄ alkyl radical; and
X₄ and X₅, independently of each other, are chosen from a hydrogen atom, or a linear C₁-C₆ alkyl radical or a branched C₃-C₆ alkyl radical, optionally ending with a radical —OX₆, wherein X₆ is chosen from a hydrogen atom or a linear C₁-C₆ or branched C₃-C₆ alkyl radical,
wherein X₄ and X₅ may form, with the nitrogen atom that bears them, a saturated or unsaturated 5- to 8-membered heterocycle, wherein at least one of the ring members may be a heteroatom chosen from O, S, or N; the heterocycle optionally substituted with at least one linear or branched C₁-C₄ alkyl, C₁-C₄ hydroxyalkyl, or dimethylamino radical.

16. The method according to claim 15, wherein R, R₂, R₃, R₄, and R₅ each represent a hydrogen atom.

17. The method according to claim 15, wherein the saturated or unsaturated 5- to 8-membered heterocycle is chosen from imidazole, pyridine, piperazine, pyrrolidine, morpholine, pyrimidine, thiazole, benzimidazole, benzothiazole, oxazole, benzotriazole, triazole, pyrazole, benzoxazole, or piperidine rings.

18. The method according to claim 15, wherein R′₁ is chosen from a radical X₄X₅N-Alk-, wherein Alk is chosen from a linear or branched C₂-C₅ alkyl chain, and X₄ and X₅, independently of each other, are chosen from a linear C₁-C₆ alkyl radical, optionally ending with a radical —OX₆, wherein X₆ is chosen from a hydrogen atom or a linear C₁-C₆ alkyl radical.

19. The method according to claim 15, wherein R′₁ is chosen from a radical X₄X₅N-Alk-, wherein Alk is chosen from a linear or branched C₂-C₅ alkyl chain, and X₄ and X₅ form, with the nitrogen atom that bears them, a saturated or unsaturated 5- to 8-membered heterocycle, wherein at least one ring member may be a heteroatom chosen from O, S, or N, chosen from imidazole, piperazine, pyrrolidine, morpholine, or piperidine rings, the heterocycle optionally substituted with at least one linear or branched C₁-C₄ alkyl, C₁-C₄ hydroxyalkyl, or dimethylamino radical.

20. The method according to claim 15, wherein:
R, R₂, R₃, R₄, and R₅ each represent a hydrogen atom,
R′₁ is chosen from a radical X₄X₅N-Alk-, wherein Alk is chosen from a linear or branched C₂-C₅ alkyl chain, and
X₄ and X₅, independently of each other, are chosen from a linear C₁-C₆ alkyl radical, optionally ending with a radical —OX₆, wherein X₆ is chosen from a hydrogen atom or a linear C₁-C₆ alkyl radical, or X₄ and X₅ may form, with the nitrogen atom that bears them, a saturated or unsaturated 5- to 8-membered heterocycle, wherein at least one of the ring members may be a heteroatom chosen from O, S, or N, chosen from imidazole, piperazine, pyrrolidine, morpholine, or piperidine rings, wherein the heterocycle is optionally substituted with at least one linear or branched C₁-C₄ alkyl, C₁-C₄ hydroxyalkyl, or dimethylamino radical.

21. A benzoxazine-based compound chosen from:
i) compounds corresponding to formula (II) below, addition salts thereof, optical isomers, geometrical isomers and tautomers thereof, or solvates thereof: 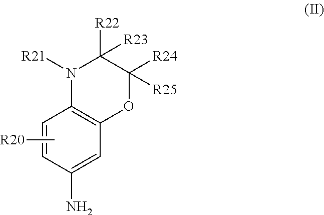
wherein:
R₂₁ is a radical chosen from:
a linear C₄-C₁₀ alkyl radical ending with a group —NX₂₁X₂₂,
a branched C₃-C₁₀ alkyl radical ending with a group —NX₂₁X₂₂,
a linear C₂-C₁₀ or branched C₃-C₁₀ alkyl radical, interrupted with at least one non-adjacent heteroatom chosen from O or S, or at least one non-adjacent group —NR′₂₀, the alkyl radical ending with a group —NX₂₁X₂₂ or —OX₂₃,
a linear C₂-C₃ alkyl radical ending with a group —NX₃₁X₃₂,
a linear C₂-C₁₀ or branched C₃-C₁₀ alkyl radical, optionally interrupted with at least one non-adjacent heteroatom chosen from O or S or at least one non-adjacent groups —NR′₂₀, the alkyl radical ending with a radical chosen from 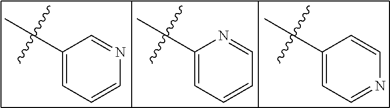
X₂₁ and X₂₂, independently of each other, are chosen from:
a hydrogen atom,
a linear C₁-C₆ alkyl radical or a branched C₃-C₆ alkyl radical, or
a linear C₁-C₆ hydroxyalkyl radical or a branched C₃-C₆ hydroxyalkyl radical,
X₂₁ and X₂₂ may form, with the nitrogen atom that bears them, a saturated or unsaturated 5- to 8-membered heterocycle, wherein at least one of the ring members may be a heteroatom chosen from O, S, or N; the heterocycle optionally substituted with at least one linear or branched C₁-C₄ alkyl, C₁-C₄ hydroxyalkyl or dimethylamino radical;
X₂₃ is chosen from a hydrogen atom or a linear C₁-C₆ or branched C₃-C₆ alkyl radical;
R′₂₀ is chosen from a hydrogen atom or a linear C₁-C₄ or branched C₃-C₄ alkyl radical;
X₃₁ and X₃₂, independently of each other, are chosen from:
a linear C₂-C₆ alkyl radical or a branched C₃-C₆ alkyl radical, or
a linear C₁-C₆ hydroxyalkyl radical or a branched C₃-C₆ hydroxyalkyl radical;
R₂₀ is chosen from a hydrogen atom or a C₁-C₄ alkyl or C₁-C₄ hydroxyalkyl radical;
R₂₂, R₂₃, R₂₄, and R₂₅, independently of each other, are chosen from a hydrogen atom or a linear C₁-C₄ or branched C₃-C₄ alkyl radical optionally substituted with a hydroxyl radical, or
ii) the following compounds: 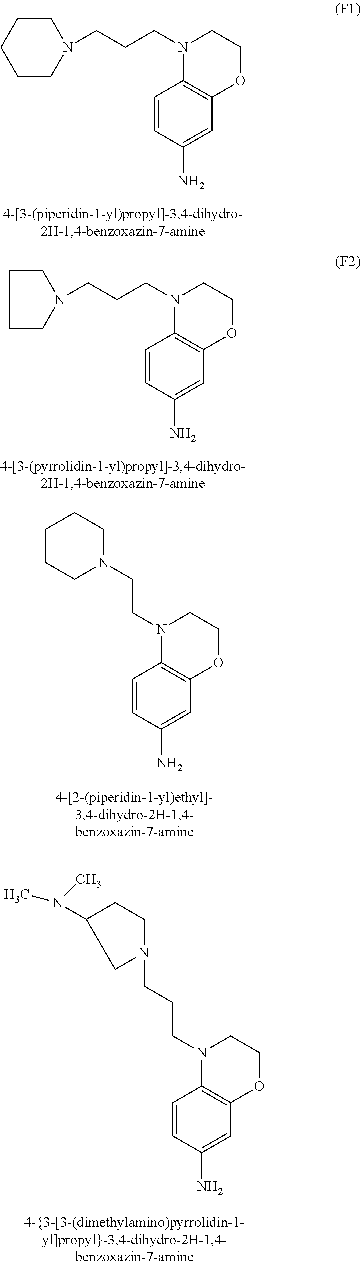 [SMILES: Nc1ccc2c(c1)OCCN2CCCN1CCCCC1 ; Nc1ccc2c(c1)OCCN2CCCN1CCCC1 ; Nc1ccc2c(c1)OCCN2CCN1CCCCC1 ; CN(C)C1CCN(CCCN2CCOc3cc(N)ccc32)C1] 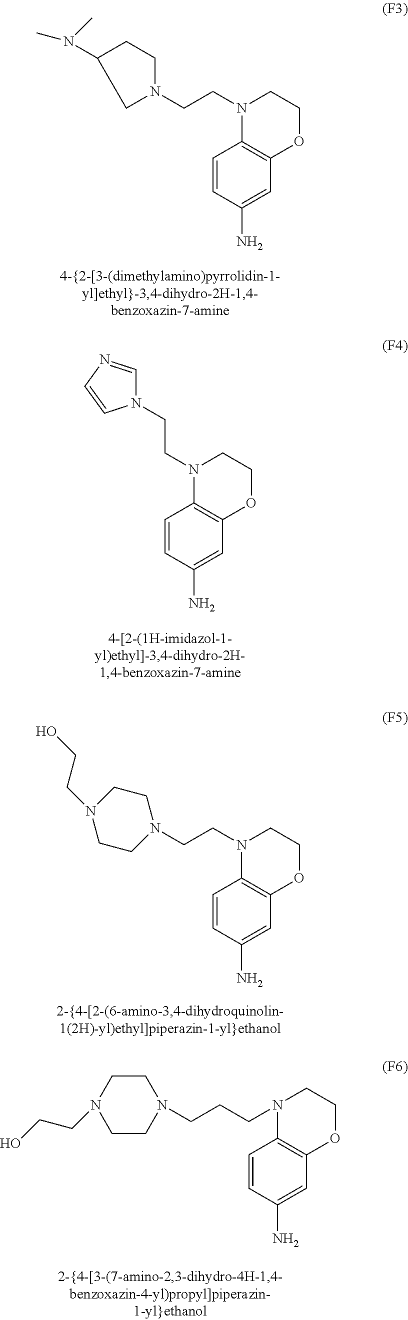 [SMILES: CN(C)C1CCN(CCN2CCOc3cc(N)ccc32)C1 ; Nc1ccc2c(c1)OCCN2CCn1ccnc1 ; Nc1ccc2c(c1)OCCN2CCN1CCN(CCO)CC1 ; Nc1ccc2c(c1)OCCN2CCCN1CCN(CCO)CC1]
or salts, solvates, or optical or geometrical isomers thereof.

22. The compound according to claim 21, wherein R₂₂, R₂₃, R₂₄, and R₂₅ each represent a hydrogen atom.

23. The compound according to claim 21, wherein R₂₁ is chosen from a linear C₂-C₃ alkyl radical ending with a group —NX₃₁X₃₂, wherein X₃₁ and X₃₂, independently of each other, are chosen from a linear C₂-C₆ alkyl radical or a branched C₃-C₆ alkyl radical, or a linear C₁-C₆ hydroxyalkyl radical or a branched C₃-C₆ hydroxyalkyl radical.

24. The compound according to claim 21, wherein the saturated or unsaturated 5- to 8-membered heterocycle is chosen from imidazole, pyridine, piperazine, pyrrolidine, morpholine, pyrimidine, thiazole, benzimidazole, benzothiazole, oxazole, benzotriazole, triazole, pyrazole, benzoxazole or piperidine rings.

25. A composition for treating keratin fibers, comprising at least one benzoxazine-based compound chosen from:
i) compounds corresponding to formula (II) below, addition salts thereof, optical isomers, geometrical isomers and tautomers thereof, or solvates thereof: 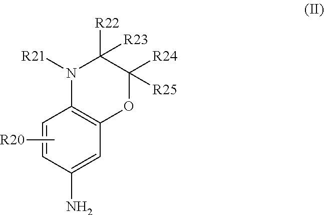
wherein:
R₂₁ is a radical chosen from:
a linear C₄-C₁₀ alkyl radical ending with a group —NX₂₁X₂₂,
a branched C₃-C₁₀ alkyl radical ending with a group —NX₂₁X₂₂,
a linear C₂-C₁₀ or branched C₃-C₁₀ alkyl radical, interrupted with at least one non-adjacent heteroatom chosen from O or S, or at least one non-adjacent group —NR′₂₀, the alkyl radical ending with a group —NX₂₁X₂₂ or —OX₂₃,
a linear C2-C3 alkyl radical ending with a group —NX₃₁X₃₂,
a linear C₂-C₁₀ or branched C₃-C₁₀ alkyl radical, optionally interrupted with at least one non-adjacent heteroatom chosen from O or S or at least one non-adjacent groups —NR′₂₀, the alkyl radical ending with a radical chosen from 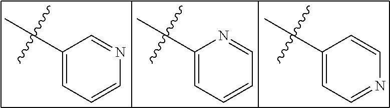
X₂₁ and X₂₂, independently of each other, are chosen from:
a hydrogen atom,
a linear C₁-C₆ alkyl radical or a branched C₃-C₆ alkyl radical, or
a linear C₁-C₆ hydroxyalkyl radical or a branched C₃-C₆ hydroxyalkyl radical,
X₂₁ and X₂₂ may form, with the nitrogen atom that bears them, a saturated or unsaturated 5- to 8-membered heterocycle, wherein at least one of the ring members may be a heteroatom chosen from O, S, or N; the heterocycle optionally substituted with at least one linear or branched C₁-C₄ alkyl, C₁-C₄ hydroxyalkyl or dimethylamino radical;
X₂₃ is chosen from a hydrogen atom or a linear C₁-C₆ or branched C₃-C₆ alkyl radical;
R′₂₀ is chosen from a hydrogen atom or a linear C₁-C₄ or branched C₃-C₄ alkyl radical;
X₃₁ and X₃₂, independently of each other, are chosen from:
a linear C₂-C₆ alkyl radical or a branched C₃-C₆ alkyl radical, or
a linear C₁-C₆ hydroxyalkyl radical or a branched C₃-C₆ hydroxyalkyl radical;
R₂₀ is chosen from a hydrogen atom or a C₁-C₄ alkyl or C₁-C₄ hydroxyalkyl radical;
R₂₂, R₂₃, R₂₄, and R₂₅, independently of each other, are chosen from a hydrogen atom or a linear C₁-C₄ or branched C₃-C₄ alkyl radical optionally substituted with a hydroxyl radical, or
ii) the following compounds: 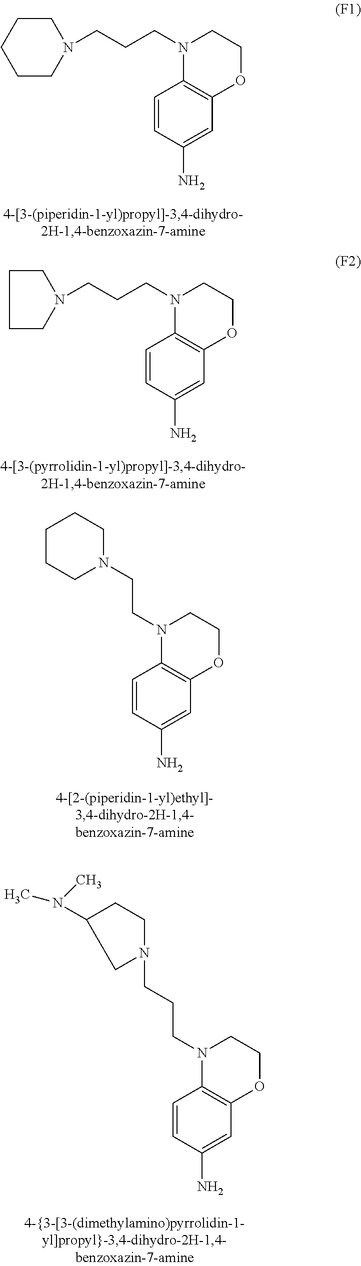 [SMILES: Nc1ccc2c(c1)OCCN2CCCN1CCCCC1 ; Nc1ccc2c(c1)OCCN2CCCN1CCCC1 ; Nc1ccc2c(c1)OCCN2CCN1CCCCC1 ; CN(C)C1CCN(CCCN2CCOc3cc(N)ccc32)C1] 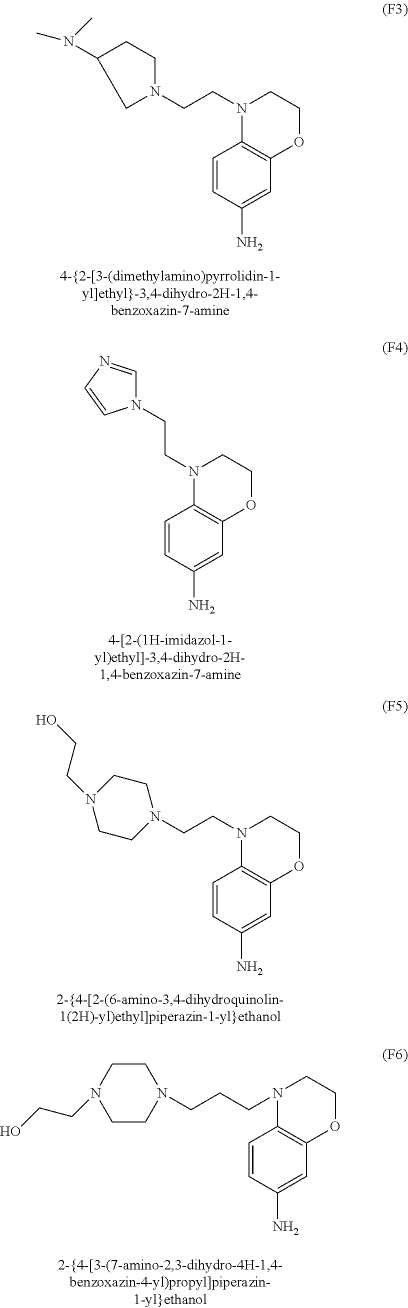 [SMILES: CN(C)C1CCN(CCN2CCOc3cc(N)ccc32)C1 ; Nc1ccc2c(c1)OCCN2CCn1ccnc1 ; Nc1ccc2c(c1)OCCN2CCN1CCN(CCO)CC1 ; Nc1ccc2c(c1)OCCN2CCCN1CCN(CCO)CC1]
or salts, solvates, or optical or geometrical isomers thereof.

26. A method for dyeing keratin fibers, comprising applying a composition to the fibers, the composition comprising at least one benzoxazine-based compound chosen from:
i) compounds corresponding to formula (II) below, addition salts thereof, optical isomers, geometrical isomers and tautomers thereof, or solvates thereof: 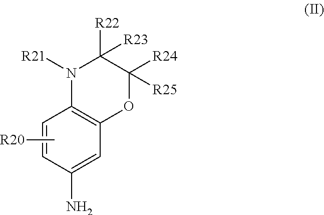
wherein:
R₂₁ is a radical chosen from:
a linear C₄-C₁₀ alkyl radical ending with a group —NX₂₁X₂₂,
a branched C₃-C₁₀ alkyl radical ending with a group —NX₂₁X₂₂,
a linear C₂-C₁₀ or branched C₃-C₁₀ alkyl radical, interrupted with at least one non-adjacent heteroatom chosen from O or S, or at least one non-adjacent group —NR′₂₀, the alkyl radical ending with a group —NX₂₁X₂₂ or —OX₂₃,
a linear C₂-C₃ alkyl radical ending with a group —NX₃₁X₃₂,
a linear C₂-C₁₀ or branched C₃-C₁₀ alkyl radical, optionally interrupted with at least one non-adjacent heteroatom chosen from O or S or at least one non-adjacent groups —NR′₂₀, the alkyl radical ending with a radical chosen from 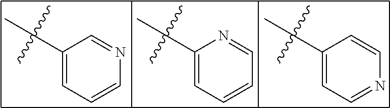
X₂₁ and X₂₂, independently of each other, are chosen from:
a hydrogen atom,
a linear C₁-C₆ alkyl radical or a branched C₃-C₆ alkyl radical, or
a linear C₁-C₆ hydroxyalkyl radical or a branched C₃-C₆ hydroxyalkyl radical,
X₂₁ and X₂₂ may form, with the nitrogen atom that bears them, a saturated or unsaturated 5- to 8-membered heterocycle, wherein at least one of the ring members may be a heteroatom chosen from O, S, or N; the heterocycle optionally substituted with at least one linear or branched C₁-C₄ alkyl, C₁-C₄ hydroxyalkyl or dimethylamino radical;
X₂₃ is chosen from a hydrogen atom or a linear C₁-C₆ or branched C₃-C₆ alkyl radical;
R′₂₀ is chosen from a hydrogen atom or a linear C₁-C₄ or branched C₃-C₄ alkyl radical;
X₃₁ and X₃₂, independently of each other, are chosen from:
a linear C₂-C₆ alkyl radical or a branched C₃-C₆ alkyl radical, or
a linear C₁-C₆ hydroxyalkyl radical or a branched C₃-C₆ hydroxyalkyl radical;
R₂₀ is chosen from a hydrogen atom or a C₁-C₄ alkyl or C₁-C₄ hydroxyalkyl radical;
R₂₂, R₂₃, R₂₄, and R₂₅, independently of each other, are chosen from a hydrogen atom or a linear C₁-C₄ or branched C₃-C₄ alkyl radical optionally substituted with a hydroxyl radical, or
ii) the following compounds: 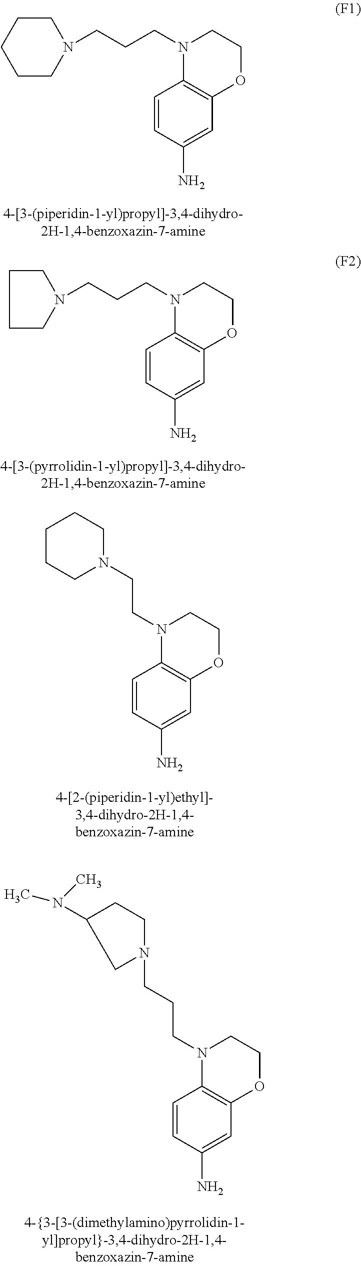 [SMILES: Nc1ccc2c(c1)OCCN2CCCN1CCCCC1 ; Nc1ccc2c(c1)OCCN2CCCN1CCCC1 ; Nc1ccc2c(c1)OCCN2CCN1CCCCC1 ; CN(C)C1CCN(CCCN2CCOc3cc(N)ccc32)C1] 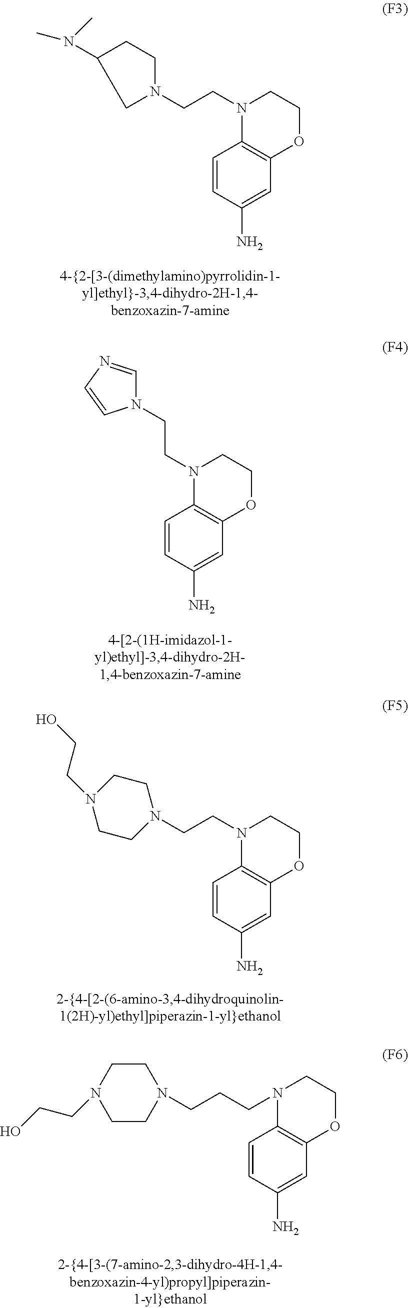 [SMILES: CN(C)C1CCN(CCN2CCOc3cc(N)ccc32)C1 ; Nc1ccc2c(c1)OCCN2CCn1ccnc1 ; Nc1ccc2c(c1)OCCN2CCN1CCN(CCO)CC1 ; Nc1ccc2c(c1)OCCN2CCCN1CCN(CCO)CC1]
or the salts, solvates, or optical or geometrical isomers thereof.
